SEQUENCE LISTING (9 sequences)

SEQ ID NO: 1 - protein, 537 aa, synthetic construct
MGTVNKPVVGVLMGFGIITGTLRITNPVRASVLRYDDFHTDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLR

SEQ ID NO: 2 - protein, 535 aa, synthetic construct
MVSAIVLYVLLAAAAHSAFARITNPVRASVLRYDDFHTDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLR

SEQ ID NO: 3 - protein, 546 aa, synthetic construct
MGTVNKPVVGVLMGFGIITGTLRITNPVRASVLRYDDFHTDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLA

SEQ ID NO: 4 - protein, 544 aa, synthetic construct
MVSAIVLYVLLAAAAHSAFARITNPVRASVLRYDDFHTDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLA

SEQ ID NO: 5 - protein, 623 aa, synthetic construct
MGTVNKPVVGVLMGFGIITGTLRITNPVRASVLRYDDFHTDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 6 - protein, 620 aa, synthetic construct
MVSAIVLYVLLAAAAHSAFAITNPVRASVLRYDDFHTDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 7 - protein, 570 aa, synthetic construct
MVSAIVLYVLLAAAAHSAFAITNPVRASVLRYDDFHTDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRLAIYSTVASSLVLVVSLGAISFWMCSNGSLQCRICI

SEQ ID NO: 8 - protein, 570 aa, synthetic construct; identical to SEQ ID NO: 7

SEQ ID NO: 9 - protein, 271 aa, synthetic construct
MFLIQCLISAVIFYIQVTNALIFKGDHVSLQVNSSLTSILIPMQNDNYTEIKGQLVFIGEQLPTGTNYSGTLELLYADTVAFCFRSVQVIRYDGCPRIRTSAFISCRYKHSWHYGNSTDRISTEPDAGVMLKITKPGINDAGVYVLLVRLDHSRSTDGFILGVNVYTAGSHHNIHGVIYTSPSLQNGYSTRALFQQARLCDLPATPKGSGTSLFQHMLDLRAGKSLEDNPWLHEDVVTTETKSVVKEGIENHVYPTDMSTLPEKSLNDPPE

(19) United States
(12) Patent Application Publication
(10) Pub. No.: US 2026/0199453 A1
(43) Pub. Date: Jul. 16, 2026

(54) IMMUNOGENIC VARICELLA ZOSTER VACCINE COMPOSITIONS

(71) Applicant: NOVAVAX, INC., Gaithersburg, MD (US)

(72) Inventors: Gale SMITH, Gaithersburg, MD (US); Dorothy JOHNSON, Gaithersburg, MD (US); Brianna YOUNG, Gaithersburg, MD (US)

(21) Appl. No.: 19/445,101

(22) Filed: Jan. 9, 2026

Related U.S. Application Data
(60) Provisional application No. 63/744,034, filed on Jan. 10, 2025.

(51) Int. Cl. A61K 39/25; A61K 39/00; A61P 37/04

(52) U.S. Cl. CPC A61K 39/25; A61P 37/04; A61K 2039/55577

(57) ABSTRACT

Disclosed herein are immunogenic compositions comprising novel varicella zoster glycoproteins, as well as methods of using the immunogenic compositions. Methods for preparing the immunogenic compositions, and methods of administering the immunogenic compositions are also disclosed.

### RELATED APPLICATION

The present application claims priority to U.S. Provisional Patent Application No. 63/744,034, filed on Jan. 10, 2025, the contents of which is incorporated by reference in its entirety.

### DESCRIPTION OF THE TEXT FILE SUBMITTED ELECTRONICALLY

This application contains a Sequence Listing which has been submitted electronically in XML format and is hereby incorporated by reference in its entirety. Said XML copy, created on Sep. 8, 2025, is named 1450_128US1_Sequence_Listing_01_09_2026 and is 13,564 bytes in size.

### FIELD OF THE INVENTION

The present invention generally relates to immunogenic compositions comprising novel varicella zoster glycoproteins, as well as methods of using the immunogenic compositions. The invention also provides methods for producing the immunogenic compositions and methods of stimulating immune responses with the compositions. The invention also provides methods of administering the immunogenic compositions.

### BACKGROUND OF THE INVENTION

The Varicella Zoster virus (VZV), also known as human herpesvirus 3 (HHV-3), is a member of the alphaherpesvirus subfamily of the Herpesviridae family of viruses. VZV is an enveloped virus with a linear double-stranded DNA genome of approximately 125,000 nucleotides. Its genome is encased by an icosahedral nucleocapsid. The tegument, located in the space between the nucleocapsid and the viral envelope, is a structure comprised of virally-encoded proteins and enzymes. The viral envelope is acquired from host cell membranes and contains viral-encoded glycoproteins.

The VZV genome, the smallest among the human herpesviruses, encodes at least 70 open reading frames, eight of which encode putative glycoproteins (gE, gI, gB, gH, gK, gL, gC, and gM) that function in different steps of the viral replication cycle. Glycoprotein E (gE, also designated ORF 68) is essential for viral replication (Mallory et al. (1997) J. Virol. 71:8279-8288; Mo et al. (2002) Virology 304:176-186) and is the most abundant glycoprotein found in infected cells as well as the mature virion (Grose, 2002, The predominant varicella-zoster virus is the gE and gI glycoprotein complex, In Structure-function relationships of human pathogenic viruses, Holzenburg and Bogner (eds.), Kluwer Academic/Plenum Publishers, New York, N.Y.). Glycoprotein I (gI, also designated ORF 67) forms a complex with gE in infected cells, which facilitates the endocytosis of both glycoproteins and directs them to the trans-Golgi where the final viral envelope is acquired (Olson and Grose (1998) J. Virol. 72:1542-1551). Glycoprotein B (gB, also designated ORF 31), thought to play a role in virus entry, binds to neutralizing antibodies and is the second most prevalent glycoprotein (reviewed in Arvin (1996) Clin. Microbiol. Rev. 9:361-381). Glycoprotein H (gH) is thought to have a fusion function facilitating cell to cell spread of the virus. Antibodies to gE, gB, and gH are prevalent after natural infection and following vaccination, and have been shown to neutralize viral infectivity in vitro (Keller et al. (1984) J. Virol. 52:293-297; Arvin et al. (1986) J. Immunol. 137:1346-1351; Vafai et al. (1988) J. Virol. 62:2544-2551; Forghani et al. (1990) J. Clin. Microbiol. 28:2500-2506).

Primary infection with VZV causes chickenpox (varicella) characterized by an extremely contagious skin rash occurring predominantly on the face and trunk. After initial infection, the viral DNA can integrate into the genome of host neuronal cells and remain dormant for several years. The virus can reactivate and cause the disease shingles (herpes zoster) in adults. Shingles produces a skin rash that is distinct from that produced during the primary infection. The rash is associated with severe pain and can result in more serious conditions, such as post-herpetic neuralgia.

A Varicella vaccine (Varivax) is available to the general public and has been added to the recommended vaccination schedule for children in several countries including the United States. Although the Varicella vaccine has proven to be safe, there is some evidence that the immunity to VZV infection conferred by the vaccine wanes over time (Chaves et al. (2007) N. Engl. J. Med. 356:1121-1129). Therefore, vaccinated individuals would remain susceptible to shingles, the more serious condition caused by VZV. In addition, the vaccine is made from live attenuated virus, which creates the possibility of an individual developing either chickenpox or shingles from the vaccination. In fact, there have been several cases of shingles reported that appeared to be caused by the strain used in the vaccine (Matsubara et al. (1995). Acta Paediatr Jpn 37:648-50; Hammerschlag et al. (1989). J Infect Dis. 160:535-7). The live attenuated virus present in the vaccine also limits the use of the vaccine in immunocompromised individuals. The development of vaccines that prevent or reduce the severity of these life-threatening infectious diseases is desirable. However, human vaccine development remains challenging because of the highly sophisticated evasion mechanisms of pathogens and difficulties stabilizing vaccines. Optimally, a vaccine must both induce antibodies that block or neutralize infectious agents and remain stable in various environments, including environments that do not enable refrigeration. Combination of two antigens from two pathogens in a single vaccine composition is particularly challenging because the antigens may interact with each other, preventing a sufficient immune response to either pathogen.

Matrix-M adjuvant enhances acquired B-cell immunity by broadening epitope recognition, increasing neutralizing antibody titers, and inducing long-lasting memory B cells in the plasma and bone marrow. Matrix-M adjuvant also induces early activation of innate cells at the injection site and draining lymph nodes, improving magnitude and quality of acquired responses (Stertman et al, The Matrix-M adjuvant: A critical component of vaccines for the 21st century. 2023. Hum Vacc Imm 19(1).). Matrix-M is known to be taken up by antigen presenting cells (APCs) where the low pH of the lysosome releases saponins, inducing lysosomal membrane destabilization and endocytosis of antigens into the cytosol. Matrix-M adjuvant offers potential for pre-pandemic priming with seasonal vaccines.

### SUMMARY OF THE INVENTION

One embodiment of the invention relates to an immunogenic composition for stimulating an immune response against varicella zoster. The composition comprises an effective amount of at least one recombinant varicella zoster virus glycoprotein, and a saponin-based adjuvant comprising Matrix-A and Matrix-C.

In some aspects, the varicella zoster virus glycoprotein comprises recombinant varicella zoster virus glycoprotein E (VZV gE).

In some aspects, the recombinant VZV gE comprises an extracellular region has at least 90% homology to the amino acid sequence as set forth in SEQ ID No: 4.

In some aspects, the varicella zoster virus glycoprotein comprises varicella zoster virus glycoprotein E (VZV gE) complexed with varicella zoster virus glycoprotein I (VZV gI).

In some aspects, the recombinant VZV gE comprises an extracellular region having at least 90% homology to the amino acid sequence as set forth in SEQ ID No: 2.

In some aspects, The immunogenic composition of claim 4, wherein the recombinant VZV gE comprises an extracellular region having at least 90% homology to the amino acid sequence as set forth in SEQ ID No: 9.

In some aspects, the saponin-based adjuvant comprises Matrix-A and Matrix-C present in a weight ratio of 85:15.

In some aspects, the saponin-based adjuvant comprises a dose of about 5 μg to about 200 μg.

In some aspects, the composition further comprises a pharmaceutically acceptable buffer.

In some aspects, a method of stimulating an immune response against varicella zoster virus includes administering the composition to a subject.

In some aspects, the composition is administered to the subject through at least one of subcutaneous, intraperitoneal, intravenous, intraarterial, intramuscular, or intrasternal injection, intravenous, or kidney dialytic infusion techniques.

In some aspects, a prefilled syringe contains the composition.

In some aspects, a kit comprises a first vial containing an effective amount of at least one recombinant varicella zoster virus glycoprotein and a second vial containing a saponin-based adjuvant comprising Matrix-A and Matrix-C.

In some aspects, the first vial containing the effective amount of at least one recombinant varicella zoster virus glycoprotein also includes space to receive the saponin-based adjuvant comprising Matrix-A and Matrix-C from the second vial.

Another embodiment of the invention is directed to an immunogenic composition for stimulating an immune response against varicella zoster, comprising an effective amount of recombinant varicella zoster virus (VZV) glycoprotein, wherein the VZV glycoprotein comprises VZV glycoprotein E (VZV gE) complexed with VZV glycoprotein I (VZV gI).

In some aspects, the VZV gE has at least 90% homology to the amino acid sequence as set forth in SEQ ID No: 2.

In some aspects, the VZV gE has at least 90% homology to the amino acid sequence as set forth in SEQ ID No: 9.

In some aspects, the composition further comprises a saponin-based adjuvant.

In some aspects, the saponin-based adjuvant comprises AS01_{B}.

In some aspects, the saponin-based adjuvant comprises Matrix-A and Matrix-C present in a weight ratio of between 80:20 and 90:10. In some aspects, the ratio is 85:15.

In some aspects, the saponin-based adjuvant is present in a dose of about 5 μg to about 200 μg.

In some aspects, the recombinant varicella zoster virus glycoprotein is present at about 0.1 μg to 200 μg.

In some aspects, the composition comprises a pharmaceutically acceptable buffer.

In some aspects, a method of stimulating an immune response against VZV comprises administering the composition to a subject.

In some aspects, the composition is administered to the subject through at least one of subcutaneous, intraperitoneal, intravenous, intraarterial, intramuscular, or intrasternal injection, intravenous, or kidney dialytic infusion techniques.

In some aspects, a prefilled syringe contains the composition.

In some aspects, a kit comprises a first vial containing the immunogenic composition and a second vial containing a saponin-based adjuvant.

In some aspects, the first vial includes space to receive contents of the second vial or the second vial includes space to receive contents of the first vial.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The invention may be more completely understood in consideration of the following detailed description of various embodiments of the invention in connection with the accompanying drawings.

FIG. 1 schematically illustrates the structures of the peptide used in the Shingrix vaccine, the BV2623 gE construct and the BV2548 gE/gI construct.

FIG. 2 shows the sequence for gE BV2623. The first 20 residues are a gp64 signal peptide. The T and L residues in bold constitute Dumas strain variations, and the last eight residues are a truncated transmembrane domain.

FIG. 3 schematically illustrates the timeline for the experiment discussed in Example 2.

FIGS. 4A and 4B show the Anti-VZV gE IgG Responses in mice at Day 35 following administration of BV2623 versus commercially available Shingrix, for anti-VZV gE (BV2623) IgG titer (FIG. 4A) and anti-Shingrix IgG titer (FIG. 4B).

FIGS. 5A and 5B show the effector memory CD4+ T cells Th1/Th2 cytokine responses in mice spleen following stimulation with BV2623 (FIG. 5A) versus peptide pool gE (FIG. 5B).

FIGS. 6A and 6B show the effector memory poly-functional CD4+ T cell triple Th1+ cytokine response, following stimulation with BV2623 (FIG. 6A) and peptide pool gE (FIG. 6B).

FIGS. 7A and 7B show the total CD4+ T cell Th1 responses in mice for BV2623+Matrix-M versus Shingrix+AS01B, following stimulation with BV2623 (FIG. 7A) versus peptide pool gE (FIG. 7B). For each bar, the lowest section represents IFNγ, the middle section represents TNFα and the top section represents IL2.

FIGS. 8A and 8B show the total CD4+ T cells Th1 Responses comparing BV2623 (0.5 μg) versus Shingrix, following stimulation with BV2623 (FIG. 8A) versus peptide pool stimulation (FIG. 8B). For each bar, the lowest section represents IFNγ, the next section represents TNFα, the second top section represents IL2 and the top section represents triple.

FIG. 9 schematically illustrates the timeline for the experiment discussed in Example 3.

FIGS. 10A and 10B present results of a serum CXCL10 (IP-10) cytokine assay taken 24 hours post-first dose (FIG. 10A) and 24 hours post-second dose (FIG. 10B) using a LEGENDplex-13 mouse anti-virus response panel.

FIGS. 11A and 11B present results of a serum CXCL1 (KC) cytokine assay taken 24 hours post-first dose (FIG. 11A) and 24 hours post-second dose (FIG. 11B) using the LEGENDplex-13 mouse anti-virus response panel.

FIGS. 12A and 12B present results of a serum MCP (CCL2) cytokine assay taken 24 hours post-first dose (FIG. 12A) and 24 hours post-second dose (FIG. 12B) using the LEGENDplex-13 mouse anti-virus response panel.

FIGS. 13A and 13B present results of a serum IFN-γ cytokine assay taken 24 hours post-first dose (FIG. 13A) and 24 hours post-second dose (FIG. 13B) using the LEGENDplex-13 mouse anti-virus response panel.

FIGS. 14A and 14B present results of a serum TNF-α cytokine assay taken 24 hours post-first dose (FIG. 14A) and 24 hours post-second dose (FIG. 14B) using the LEGENDplex-13 mouse anti-virus response panel.

FIGS. 15A and 15B present results of a serum IL-6 cytokine assay taken 24 hours post-first dose (FIG. 15A) and 24 hours post-second dose (FIG. 15B) using the LEGENDplex-13 mouse anti-virus response panel.

FIGS. 16A and 16B present results of a serum CCL5 (RANTES) cytokine assay taken 24 hours post-first dose (FIG. 16A) and 24 hours post-second dose (FIG. 16B) using the LEGENDplex-13 mouse anti-virus response panel.

FIGS. 17A and 17B present results of a serum IFN-α cytokine assay taken 24 hours post-first dose (FIG. 17A) and 24 hours post-second dose (FIG. 17B) using the LEGENDplex-13 mouse anti-virus response panel.

FIGS. 18A and 18B present results of a serum IL-1β cytokine assay taken 24 hours post-first dose (FIG. 18A) and 24 hours post-second dose (FIG. 18B) using the LEGENDplex-13 mouse anti-virus response panel.

FIGS. 19A and 19B show contour plots of reactogenicity induced by various VZV vaccines and placebo for nine different proinflammatory cytokines 24 hours after the first does (FIG. 19A) and 24 hours after the second dose (FIG. 19B)

FIG. 20 shows the CD4⁺ T cell response following stimulation using the VZV gE peptide pool for four different cytokines, IFNγ, TNFα, IL2 and IL4, across all mouse groups. The nine bars under each cytokine correspond to mouse groups 1-9 in order.

FIG. 21 shows the CD8⁺ T cell response following stimulation using the VZV gE peptide pool for three different cytokines, IFNγ, IL2 and IL4, across all mouse groups. The nine bars under each cytokine correspond to mouse groups 1-9 in order.

FIG. 22 shows Triple Th1 CD4⁺ Th1 responses following stimulation using the VZV gE peptide pool, measured 14 days after the second immunization, for each of the 9 mouse groups.

FIGS. 23A-23C show a summary of antigen-specific CD8+ T cell responses measured 14 days after the second immunization, for Shingrix (gE)+AS01_{B}, BV2623 (gE)+Matrix-M, BV2548 (gE/gI)+Matrix-M, and placebo. FIG. 23A shows the percentage of 41BB+CD69+ in effector CD8+ T cells. FIG. 23B shows the percentage of CD25+CD69+ in effector CD8+ T cells. FIG. 23C shows the percentage of IFN-γ+CD69+ in effector CD8+ T cells.

FIGS. 24A-24O show Anti-VZV gE or gE/gI IgG titers at different points throughout the experiment, for each of the mouse groups unless indicated. FIGS. 24A and 24B respectively show anti-VZV gE and anti-VZV gE/gI IgG titers in serum after one immunization dose (Day 20). FIGS. 24C and 24D respectively show anti-VZV gE and anti-VZV gE/gI IgG titers in serum after two immunization doses (Day 35). FIG. 24E shows anti-Shingrix gE IgG titers in serum after two immunization doses (Day 35). FIG. 24F shows anti-VZV gE IgG titers in serum after two immunization doses (Day 35), while FIG. 24G shows anti-VZV gE IgG2c titers in serum after two immunization doses (Day 35) for mouse groups 1, 3, 5, 7, and 9. FIG. 24H shows anti-VZV gE IgG titers in serum after two immunization doses (Day 56), while FIG. 24I shows anti-VZV gE IgG2c titers in serum after two immunization doses (Day 56) for mouse groups 1, 3, 5, 7, and 9. FIG. 24J shows anti-VZV gE/gI IgG titers in serum after two immunization doses (Day 56) across all mouse groups while FIG. 24K shows anti-Shingrix (gE) IgG titers in serum after two immunization doses (Day 56) across all mouse groups. FIG. 24L shows anti-VZV gE IgG titers across all mouse groups at Day 112, 114, 119, or 121, while FIG. 24M shows anti-VZV gE IgG2c titers for mouse groups 1, 3, 5, 7, and 9 at Day 112/114. FIG. 24N shows anti-VZV gE/gI IgG titers in serum after two immunization doses (Day 112/114/119/121 across all mouse groups while FIG. 24O shows anti-Shingrix (gE) IgG titers in serum after two immunization doses (Day 112/114/119/121 across all mouse groups.

FIGS. 25A-25C show the time development and durability of IgG arising from various antigens as function of time over the experimental period, for mouse groups 1 (MG1), 3 (MG3), 5 (MG5), and 7 (MG7) (5 μg doses of antigen). FIG. 25A shows the anti-VZV gE IgG titer. FIG. 25B shows the anti-VZV gE/gI IgG titer. FIG. 25C shows the anti-Shingrix IgG titer.

FIG. 26 shows the number of triple Th1+ cells per 10⁶ CD4+ T cells over all mouse groups when stimulated with the VZV gE peptide pool at 11 weeks after the second dose.

FIGS. 27A and 27B show the numbers of antigen-specific bone marrow-derived memory B cells obtained using B cell ELISPOT at 11 weeks after the second immunization, over all mouse groups. FIG. 27A shows the results coated with BC2623 (VZV gE) and FIG. 27B shows the results coated with Shingrix.

FIG. 28 schematically illustrates the timeline for the experiment discussed in Example 4, to explore dose adjustment of gE/gI.

FIGS. 29A-29C show results of the immune response in the spleen. FIG. 29A shows the number of triple Th1 CD4+ T cells following stimulation with the VZV gE peptide pool, across all mouse groups. FIG. 29B shows the number of Th1/Th2 cytokines per 10⁶ CD4+ T cells for IFNγ, TNFα, IL2, and IL4, following stimulation with the VZV gE peptide pool. FIG. 29C illustrates the CD8+ T cell response by showing the percentage of IFNγ+CD69+ following stimulation with the VZV gE peptide pool.

FIGS. 30A-30F show IgG titers in serum. FIG. 30A shows anti-VZV gE (BV2623) IgG titers after one dose (day 20) and FIG. 30B shows anti-VZV gE/gI (BV2548) IgG titers after one dose (day 20). FIG. 30C shows anti-VZV gE (BV2623) IgG titers after two doses (day 33) and FIG. 30D shows anti-VZV (BV2623) IgG2c titers after two doses (day 33). FIG. 30E shows anti-VZV gE/gI (BV2548) IgG titers after two doses (day 33) and FIG. 30F shows anti-Shingrix IgG titers after two doses (day 33).

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE INVENTION

### Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “adjuvant” refers to a compound or substance that, when used in combination with an immunogen, augments or otherwise alters or modifies the immune response induced against the immunogen. Modification of the immune response may include intensification or broadening the specificity of either or both antibody and cellular immune responses.

As used herein, the term “about” or “approximately” when preceding a numerical value indicates the value plus or minus a range of 10%. For example, “about 100” encompasses 90 and 110. When applied to a range, “about” indicates the lower value of the range minus 10%, and the upper value of the range plus 10%. For example, “a range of about 100 to 200” encompasses a range of 90 (lower value-10%) to 220 (upper value+10%).

As used herein, the terms “immunogen,” “antigen,” and “epitope” refer to substances such as proteins, including glycoproteins, and peptides that are capable of eliciting an immune response.

As used herein, an “immunogenic composition” is a composition that comprises an antigen where administration of the composition to a subject results in the development in the subject of a humoral and/or a cellular immune response to the antigen.

As used herein, a “subunit” composition, for example a vaccine, includes one or more selected antigens, but not all antigens, from a pathogen. Such a composition is substantially free of intact virus or the lysate of such cells or particles and is typically prepared from at least partially purified, often substantially purified immunogenic polypeptides from the pathogen. The antigens in the subunit composition disclosed herein are typically prepared recombinantly, often using a baculovirus system.

As used herein, “substantially” refers to isolation of a substance (e.g. a compound, polynucleotide, or polypeptide) such that the substance forms the majority percent of the sample in which it is contained. For example, in a sample, a substantially purified component comprises 85%, preferably 85%-90%, more preferably at least 95%-99.5%, and most preferably at least 99% of the sample. If a component is substantially replaced the amount remaining in a sample is less than or equal to about 0.5% to about 10%, preferably less than about 0.5% to about 1.0%.

The terms “treat,” “treatment,” and “treating,” as used herein, refer to an approach for obtaining beneficial or desired results, for example, clinical results. For the purposes of this disclosure, beneficial or desired results may include inhibiting or suppressing the initiation or progression of an infection or a disease; ameliorating, or reducing the development of, symptoms of an infection or disease; or a combination thereof.

“Prevention,” as used herein, is used interchangeably with “prophylaxis” and can mean complete prevention of an infection or disease, or prevention of the development of symptoms of that infection or disease; a delay in the onset of an infection or disease or its symptoms; or a decrease in the severity of a subsequently developed infection or disease or its symptoms.

As used herein an “effective dose” or “effective amount” refers to an amount of an immunogen sufficient to induce an immune response that reduces at least one symptom of pathogen infection. An effective dose or effective amount may be determined e.g., by measuring amounts of neutralizing secretory and/or serum antibodies, e.g., by plaque neutralization, complement fixation, enzyme-linked immunosorbent (ELISA), or microneutralization assay.

As used herein, the term “vaccine” refers to an immunogenic composition, such as an immunogen derived from a pathogen, which is used to induce an immune response against the pathogen that provides protective immunity (e.g., immunity that protects a subject against infection with the pathogen and/or reduces the severity of the disease or condition caused by infection with the pathogen). The protective immune response may include formation of antibodies and/or a cell-mediated response. Depending on context, the term “vaccine” may also refer to a suspension or solution of an immunogen that is administered to a subject to produce protective immunity.

As used herein, the term “subject” includes humans and other animals. Typically, the subject is a human. For example, the subject may be an adult, a teenager, a child (2 years to 14 years of age), an infant (birth to 2 year), or a neonate (up to 2 months). In particular aspects, the subject is up to 4 months old, or up to 6 months old. In some aspects, the adults are seniors about 65 years or older, or about 60 years or older. In some aspects, the subject is a pregnant woman or a woman intending to become pregnant. In other aspects, subject is not a human; for example a non-human primate; for example, a baboon, a chimpanzee, a gorilla, or a macaque. In certain aspects, the subject may be a pet, such as a dog or cat.

As used herein, the term “pharmaceutically acceptable” means being approved by a regulatory agency of a U.S. Federal or a state government or listed in the U.S. Pharmacopeia, European Pharmacopeia or other generally recognized pharmacopeia for use in mammals, and more particularly in humans. These compositions can be useful as a vaccine and/or antigenic compositions for inducing a protective immune response in a vertebrate.

The term “polypeptide” covers a molecule comprising two or more amino acids connected via peptide bonds. Proteins are considered to be polypeptides.

### VZV Vaccine Antigens

The present disclosure provides, in some embodiments, immunogenic compositions that comprise at least one varicella zoster virus (VZV) protein. In one embodiment, the VZV protein is recombinant and comprises the ectodomain of a VZV protein and the transmembrane and/or cytoplasmic domain of a heterologous protein. In another embodiment, the VZV protein is selected from the group consisting of gE, gI, gB, gH, gK, gL, gC, and gM. In another embodiment, the VZV protein is gE and/or gI. The gE and gI glycoproteins are considered to be useful as antigens for vaccination, because gE and gI form a heterodimer found on the surface of the VZV envelope that mediates efficient cell-to-cell spread.

The invention also encompasses variants of the VZV proteins. The variants may contain alterations in the amino acid sequences of the constituent proteins. The term “variant” with respect to a polypeptide refers to an amino acid sequence that is altered by one or more amino acids with respect to a reference sequence. The variant can have “conservative” changes, wherein a substituted amino acid has similar structural or chemical properties, e.g., replacement of leucine with isoleucine. Alternatively, a variant can have “nonconservative” changes, e.g., replacement of a glycine with a tryptophan. Analogous minor variations can also include amino acid deletion or insertion, or both. Guidance in determining which amino acid residues can be substituted, inserted, or deleted without eliminating biological or immunological activity can be found using computer programs well known in the art, for example, DNASTAR software.

In certain embodiments, the VZV protein is truncated with the C-term membrane domain removed. The recombinant VZV protein further has a non-native signal peptide from the baculovirus protein gp64.

Methods of cloning VZV proteins of the invention are known in the art. For example, the gene encoding a specific VZV protein can be isolated by RT-PCR from polyadenylated mRNA extracted from cells which had been infected with a VZV virus. The resulting product gene can be cloned as a DNA insert into a vector. The term “vector” refers to the means by which a nucleic acid can be propagated and/or transferred between organisms, cells, or cellular components.

Vectors include plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. In many, but not all, common embodiments, the vectors of the present invention are plasmids.

In some embodiments of the invention proteins may comprise, mutations containing alterations which produce silent substitutions, additions, or deletions, but do not alter the properties or activities of the encoded protein or how the proteins are made. Nucleotide variants can be produced for a variety of reasons, e.g., to optimize codon expression for a particular host (change codons in the human mRNA to those preferred by insect cells such as Sf9 cells).

In addition, the nucleotides can be sequenced to ensure that the correct coding regions were cloned and do not contain any unwanted mutations. The nucleotides can be subcloned into an expression vector (e.g. baculovirus) for expression in any cell. The above is only one example of how the VZV viral proteins can be cloned. A person with skill in the art would understand that additional methods are available and are possible.

The invention also provides for constructs and/or vectors that comprise VZV nucleotides that encode for VZV proteins, including gE, gI, gM, gH, gB, tegument proteins, chimeric proteins or portions thereof. The vector may be, for example, a phage, plasmid, viral, or retroviral vector. The constructs and/or vectors that comprise VZV genes, including gE, gI, gM, gH, gB tegument proteins, their chimerics, or portions thereof, should be operably linked to an appropriate promoter, such as the AcMNPV polyhedrin promoter (or other baculovirus), phage lambda PL promoter, the E. coli lac, phoA and tac promoters, the SV40 early and late promoters, and promoters of retroviral LTRs are non-limiting examples. Other suitable promoters will be known to the skilled artisan depending on the host cell and/or the rate of expression desired. The expression constructs will further contain sites for transcription initiation, termination, and, in the transcribed region, a ribosome-binding site for translation. The coding portion of the transcripts expressed by the constructs will preferably include a translation initiating codon at the beginning and a termination codon appropriately positioned at the end of the polypeptide to be translated.

### Production of Antigen

The antigens of the present disclosure are non-naturally occurring products which do not occur in nature. The antigens contained in the nanoparticles are typically produced by recombinant expression in host cells. Standard recombinant techniques may be used, as discussed above. In embodiments, the polypeptide antigens are expressed in insect host cells using a baculovirus system. In embodiments, the baculovirus is a cathepsin-L knock-out baculovirus, or a chitinase knock-out baculovirus. Optionally, the baculovirus is a double knock-out for both cathepsin-L and chitinase. High level expression may be obtained in insect cell expression systems. Non limiting examples of insect cells are, Spodoptera frugiperda (Sf) cells, e.g. Sf9, Sf21, Trichoplusiani cells, e.g. High Five cells, and Drosophila S2 cells. In embodiments, the gE and gE/gI polypeptides described herein are produced in any suitable host cell. In embodiments, the host cell is an insect cell. In embodiments, the insect cell is an Sf9 cell.

Typical transfection and cell growth methods can be used to culture the cells. Vectors, e.g., vectors comprising polynucleotides that encode VZV glycoproteins, can be transfected into host cells according to methods well known in the art. For example, introducing nucleic acids into eukaryotic cells can be achieved by calcium phosphate co-precipitation, electroporation, microinjection, lipofection, and transfection employing polyamine transfection reagents. In one embodiment, the vector is a recombinant baculovirus. Where a single polypeptide (glycoprotein), such as gE, is being produced, the baculovirus contains a recombinant DNA sequence for encoding the glycoprotein and a suitable promoter. Where two polypeptides are produced, for example a gE/gI heterodimer, the baculovirus contains respective DNA sequences for encoding the two different polypeptides, along with two suitable promoters, one for each polypeptide. The two polypeptides are co-produced and co-purified.

Methods to grow host cells include, but are not limited to, batch, batch-fed, continuous and perfusion cell culture techniques. Cell culture means the growth and propagation of cells in a bioreactor (a fermentation chamber) where cells propagate and express protein (e.g. recombinant proteins) for purification and isolation. Typically, cell culture is performed under sterile, controlled temperature and atmospheric conditions in a bioreactor. A bioreactor is a chamber used to culture cells in which environmental conditions such as temperature, atmosphere, agitation and/or pH can be monitored. In one embodiment, the bioreactor is a stainless steel chamber. In another embodiment, the bioreactor is a pre-sterilized plastic bag (e.g. Cellbag®, Wave Biotech, Bridgewater, N.J.). In other embodiments, the pre-sterilized plastic bags are about 50 L to 3500 L bags.

After growth of the host cells, the glycoprotein may be harvested using various purification protocols. In some embodiments, the glycoprotein may be purified from the growth media using, for example chromatographic approaches, such as anion exchange, lentilectin affinity chromatography, and cation exchange.

### Adjuvants

As is also well known in the art, the immunogenicity of a particular composition can be enhanced by the use of non-specific stimulators of the immune response, known as adjuvants. The term “adjuvant” refers to a compound that, when used in combination with a specific immunogen (e.g. a VLP) in a formulation, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses. Adjuvants have been used experimentally to promote a generalized increase in immunity against unknown antigens (e.g., U.S. Pat. No. 4,877,611). Immunization protocols have used adjuvants to stimulate responses for many years, and as such, adjuvants are well known to one of ordinary skill in the art. Some adjuvants affect the way in which antigens are presented. For example, the immune response is increased when protein antigens are precipitated by alum. Emulsification of antigens also prolongs the duration of antigen presentation. The inclusion of any adjuvant described in Vogel et al., “A Compendium of Vaccine Adjuvants and Excipients (2^{nd} Edition),” herein incorporated by reference in its entirety for all purposes, is envisioned within the scope of this invention.

Adjuvants containing saponin can also be combined with the recombinant proteins disclosed herein. Saponin-based adjuvants, i.e. adjuvants containing saponin, may also be used. Saponins are glycosides extracted from the bark of the Quillaja saponaria Molina tree. These bark extracts contain a heterogeneous mixture of hundreds of related saponins with structurally different glycosylation or acylation patterns that also affect their biological activities.

In some embodiments, the compositions disclosed herein may be combined with one or more adjuvants to enhance an immune response. In other embodiments, the compositions are prepared without adjuvants, and are thus available to be administered as adjuvant-free compositions. Advantageously, adjuvant-free compositions disclosed herein may provide protective immune responses when administered as a single dose. Alum-free compositions that induce robust immune responses are especially useful in adults about 60 and older.

Saponins are a large family of plant-derived glycoconjugates that share a triterpene structure with a variety of glycoside side chains. Saponins have traditionally been used for making soaps based on being amphipathic. Saponins now also are used for making adjuvants based on having potent immune-stimulating properties, as taught, for example by Kensil et al., U.S. Pat. No. 5,057,540.

Saponins extracted from the bark of the South American soapbark tree Quillaja saponaria Molina contain a complex heterogeneous mixture of closely related saponins with structurally different glycosylation or acylation patterns that affect their biological activities. Quillaja saponaria Molina saponins can have a high degree of glycosyl O-acylation, a low degree of glycosyl O-acylation, or no glycosyl O-acylation in their naturally occurring forms. Saponins also can be chemically modified, for example by partial or complete deacylation or degradation.

Saponins of Quillaja saponaria Molina in particular can have potent adjuvant activity, but also can be chemically unstable, show hemolytic activity, and be associated with immediate pain at injection sites. Saponin preparations based on defined compositions of purified saponin fractions of Quillaja saponaria Molina are described, for example, by Cox et al., PCT/AU1995/000670 (WO96011711).

Incorporation of saponins of Quillaja saponaria Molina into particles comprising saponin and lipid can attenuate the chemical instability, hemolytic activity, and immediate pain when injected associated with saponins. Specific examples of particles comprising saponin and lipid include saponin based particles and saponin based antigen-presenting particles, as taught, for example, by Stertman et al., Human Vaccines & Immunotherapeutics, 2023, 19(1):2189885, GSK's Liposome-based Adjuvant System 01 particles, as described, for example, in Didierlaurent et al., Expert Review of Vaccines, 2017, 16(1):55-63, and Army Liposome Formulation Q particles, as described, for example, by Alving et al., Expert Review of Vaccines, 2020, 19(3):279-292.

Novavax's Matrix particle adjuvant is a saponin-based adjuvant derived from Quillaja saponins, which are extracted from the bark of the Quillaja saponaria Molina tree. The bark extract is fractionated, and fractionated saponins are formulated with phosphatidylcholine and cholesterol into cage-like Matrix particles. The Matrix adjuvant may be a formulation that is manufactured using two extracts of saponins of Quillaja saponaria Molina, termed saponin fraction A and saponin fraction C, which are described in detail below. To make a Matrix adjuvant, saponin fraction A and saponin fraction C are separately mixed with cholesterol and phosphatidylcholine, in the presence of the detergent Mega-10 to form dispersions of approximately 40-50 nm-sized stable cage-like structures, designated Matrix-A and Matrix-C particles. The Matrix-A particles may comprise fraction A but not fraction C, whereas the Matrix-C particles may comprise fraction C but not fraction A. The Mega-10 detergent is removed by diafiltration. In one embodiment, the Matrix-A to Matrix-C weight ratio is between 80:20 and 90:10. In some embodiments the Matrix-A to Matrix-C ratio is around 85:15. A specific embodiment denoted Matrix-M constitutes Matrix-A and Matrix-C particles at a fixed weight ratio of 85:15 of Matrix-A particles to Matrix-C particles. Considering saponins in more detail, as noted above, saponin preparations based on defined compositions of purified saponin fractions of Quillaja saponaria Molina are described, for example, by Cox et al., PCT/AU1995/000670 (WO96011711)

Over the years the process for fractionation of saponin raw material into fraction A and fraction C has been developed and scaled-up. For example, as described in Cox et al., PCT/AU1995/000670 (WO96011711), fractions A, B, and C can be prepared from the lipophilic fraction obtained on chromatographic separation of the crude aqueous Quillaja Saponaria Molina extract on a SEP-PAK column and elution with 70% acetonitrile in water to recover the lipophilic fraction. This lipophilic fraction can then be separated by semipreparative HPLC with elution using a gradient of from 25% to 60% acetonitrile in acidic water. Fraction A is the fraction that is eluted at approximately 39% acetonitrile. Fraction C is the fraction that is eluted at approximately 49% acetonitrile.

As noted above, saponin preparations based on defined compositions of purified saponin fractions of Quillaja saponaria Molina are described, for example, by Cox et al., PCT/AU1995/000670 (WO96011711).

In specific embodiments, Saponin Fraction C has a purity of at least 80%, as determined by HPLC. In specific embodiments, Cholesterol is derived from a plant-based source (phytosterol). In specific embodiments, Phosphatidylcholine comprises POPC fatty acid chains. Matrix particles can be made as described, for example, in Morein et al., PCT/SE1989/000528, and Morein et al., PCT/SE2003/001180.

In specific embodiments, the cage-like structure is confirmed by transmission electron microscopy (TEM).

### Formulations

The vaccine is prepared in a formulation for delivery to a subject, typically a human subject. The formulation may be aqueous-based, but it not so restricted. In some embodiments the vaccine, comprising the antigen and adjuvant, is in a buffered solution. For example, the buffer may include any pharmaceutically acceptable buffer, such as Tris or MES. The formulation may also include other excipients, such as sodium chloride (NaCl) and sucrose, and may also contain other excipients, e.g. arginine. One exemplary embodiment of formulation is 25 mM MES, 160 mM NaCl, pH 6.5 buffer. Another exemplary embodiment of formulation is 25 mM Tris, 40 mM NaCl, 150 mM Arginine, 3% (w/v) sucrose, pH 7.4. Other formulations may be used with a pH in the range 6.5-7.4.

A dose of VZV vaccine for human administration includes a therapeutically effective amount of recombinant VZV glycoprotein, likely in the range 0.5-200 μg, preferably in the range 10-100 μg. The dose of VZV glycoprotein may be in the range of about 5 μg to about 25 μg, about 1 μg to about 300 μg, about 90 μg to about 270 μg, about 100 μg to about 160 μg, about 110 μg to about 150 μg, about 120 μg to about 140 μg, or about 140 μg to about 160 μg. In embodiments, the dose of VZV glycoprotein is about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21, about 22, about 23, about 24, about 25 μg, about 26 μg, about 27 μg, about 28 μg, about 29 μg, about 30 μg, about 40 μg, about 50, about 60, about 70, about 80, about 90 about 100 μg, about 110 μg, about 120 μg, about 130 μg, about 140 μg, about 150 μg, about 160 μg, about 170 μg, about 180 μg, about 190 μg, about 200 μg, about 210 μg, about 220 μg, about 230 μg, about 240 μg, about 250 μg, about 260 μg, about 270 μg, about 280 μg, about 290 μg, or about 300 μg, including all values and ranges in between. In embodiments, the dose of VZV glycoprotein protein is 5 μg. In embodiments, the dose of VZV glycoprotein protein is 25 μg. In some embodiments, the dose of VZV glycoprotein is the same for an initial dose and for one or more subsequent boost doses.

In embodiments, it includes a therapeutically effective amount of a saponin-based adjuvant in the range 0.5-200 μg, preferably in the range 10-100 μg. In embodiments, the dose of the adjuvant administered with a recombinant VZV glycoprotein is from about 1 μg to about 100 μg, for example, about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21, about 22, about 23, about 24, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, or about 100 μg of adjuvant. In embodiments, the dose of adjuvant is about 50 μg.

### Administration

Compositions disclosed herein may be administered via a systemic route or a mucosal route or a transdermal route or directly into a specific tissue. As used herein, the term “systemic administration” includes parenteral routes of administration. In particular, parenteral administration includes subcutaneous, intraperitoneal, intravenous, intraarterial, intramuscular, or intrasternal injection, intravenous, or kidney dialytic infusion techniques. Typically, the systemic, parenteral administration is intramuscular injection. As used herein, the term “mucosal administration” includes oral, intranasal, intravaginal, intra-rectal, intra-tracheal, intestinal and ophthalmic administration. Preferably, administration is intramuscular.

The compositions disclosed herein may be stored in a pre-filled syringe. In other embodiments, the compositions may be provided in a kit that has a first vial containing the antigen and a second vial containing the adjuvant, for example a Matrix-M adjuvant. In such a case one of the vials has sufficient empty space that the contents of the other vial may be transferred in. the first vial of the kit may contain the antigen in a lyophilized form.

The VZV vaccine may be administered to a human in a single dose, or may be administered in multiple doses. In embodiments, a second dose may be administered up to one week, two weeks, three weeks, four weeks, six weeks, two months, three months, four months, five months, six months, seven months, eight months, nine months, ten months, eleven months or twelve months after the initial dose. In other embodiments a second dose may be administered within a range of time after the initial dose, for example within one week to one month, two months, three months, four months, five months, six months, seven months, eight months, nine months, ten months, eleven months or twelve months after the initial dose. In other embodiments a second dose may be administered within one month to two months, three months, four months, five months, six months, seven months, eight months, nine months, ten months, eleven months or twelve months after the initial dose. In other embodiments a second dose may be administered within two months to three months, four months, five months, six months, seven months, eight months, nine months, ten months, eleven months or twelve months after the initial dose. In other embodiments a second dose may be administered within three months and four months, five months, six months, seven months, eight months, nine months, ten months, eleven months or twelve months after the initial dose. In other embodiments a second dose may be administered within six months and seven months, eight months, nine months, ten months, eleven months or twelve months after the initial dose.

### EXAMPLES

### Example 1: VZV Secreted Glycoproteins gE and gE/gI

Using the techniques described above, the following recombinant VZV gE antigens were created.

| SEQ ID NO: | Sequence description |
|---|---|
| 1 | BV2622_gE(1-537) |
| 2 | BV2628_gp64sp.gE(23-537) |
| 3 | BV2624_gE(1-546) |
| 4 | BV2623_gp64sp.gE(23-546) |
| 5 | BV2629_gE_Dumas |
| 6 | BV2775_gp64sp.gE(24-623) |
| 7 | BV2791_ gp64sp.gE-AWis-TMCT |
| 8 | BV2792_VZV-TM-AWis-CT |
| 9 | gI(1-271) (gI portion of BV2548) |

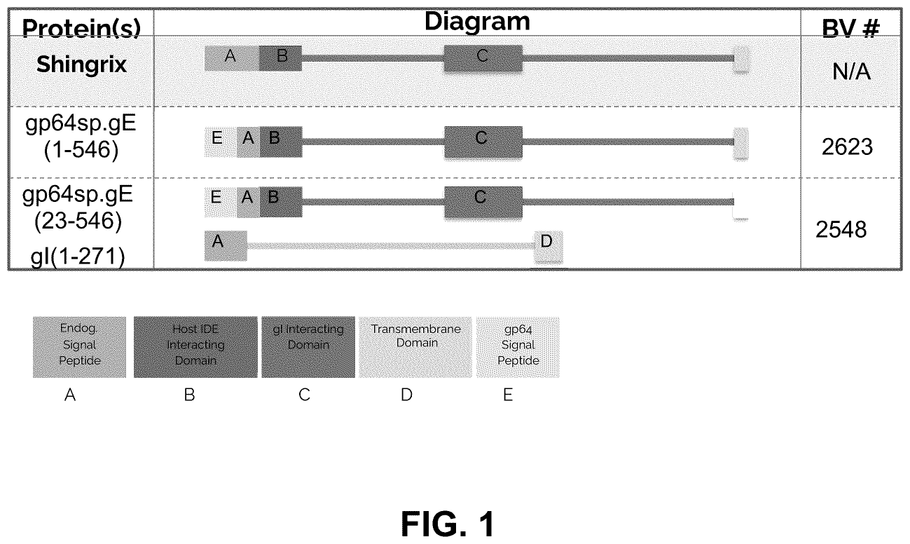

A comparison of sequence BV2623 with the Shingrix antigen is shown in FIG. 1. The Shingrix gE antigen peptide, includes an endogenous signal peptide, a host IDE interacting domain, a gI interacting domain and a transmembrane domain. The Shingrix polypeptide is based on an Oka strain gE. The BV2623 gE sequence, SEQ ID. NO: 4, starts with a gp64 signal peptide, and includes a truncated endogenous peptide, a host IDE interacting domain, a gI interacting domain, and a transmembrane domain. The BV2623 sequence is based on a Dumas strain gE.

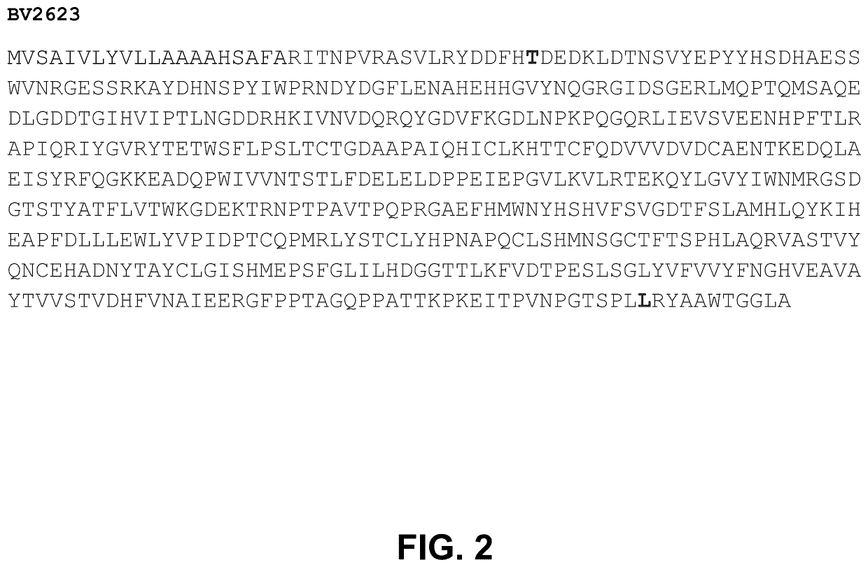

FIG. 2 shows the residue sequence for the 2623 sequence. The first 20 residues constitute the gp64 signal peptide. The T and L residues in bold show the Dumas strain variations. The last eight residues constitute a truncated transmembrane domain.

Sequence BV2548 is a gE/gI antigen, including peptide sequences of both gE and gI in a heterodimer form. The gE sequence of BV2548 is SEQ ID. NO: 2, and is similar to SEQ ID NO:4, but lacks the transmembrane domain. The gI sequence of BV2548 is SEQ ID. NO: 9.

### Example 2: Comparative Immunogenicity of New gE Construction and GSK Shingrix Vaccines in Mice

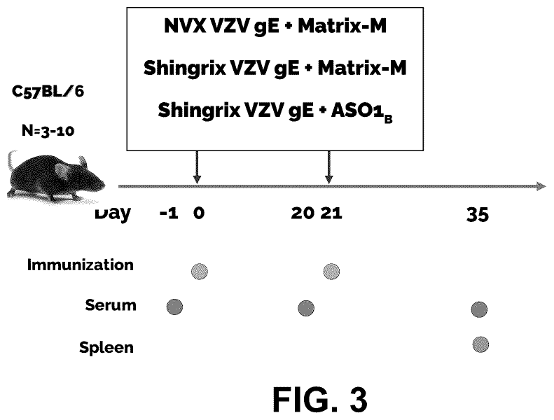

A study was made comparing the immunogenicity of the 2623 construction to that of GSK's Shingrix vaccine, in mice. The timeline of the study is shown in FIG. 3. C57BL/6 mice were administered the vaccine on days 0 and 21 of the study. Serum was taken from the mice on days-1, 20, and 35. On day 35, the serum from six mice per group and from the placebo group was collected and used in the measurement of IgG responses, using an enzyme-linked immunosorbent assay (ELISA), and CD4+ and CD8+ T cell responses, using intracellular cytokine staining (ICCS).

The table below shows the number of groups of mice in each group and what they were administered.

TABLE 1
Combinations of vaccine (antigen) and adjuvant
used in the experiment of Example 2

| | | | Vaccine | | Adjuvant |
|---|---|---|---|---|---|
| Group | N | Vaccine | Dose (μg) | Adjuvant | Dose (μg) |
| 1 | 10 | 2623 (gE) | 5 | Matrix-M | 5 |
| 2 | 10 | 2623 (gE) | 0.5 | Matrix-M | 5 |
| 3 | 10 | Shingrix (gE) | 5 | Matrix-M | 5 |
| 4 | 10 | Shingrix (gE) | 0.5 | Matrix-M | 5 |
| 5 | 10 | Shingrix (gE) | 5 | AS01_{B} | 10 |
| 6 | 10 | Shingrix (gE) | 0.5 | AS01_{B} | 10 |
| 7 | 3 | Placebo | | — | — |

Shingrix is supplied with the antigen and adjuvant in separate vials, with the expectation that the adjuvant is added to the antigen prior to administration. This procedure was used for preparing the vaccine administered to Groups 5 and 6. For Groups 3 and 4, the Shingrix antigen was mixed with Matrix-M adjuvant prior to administration, rather than the AS01_{B} adjuvant.

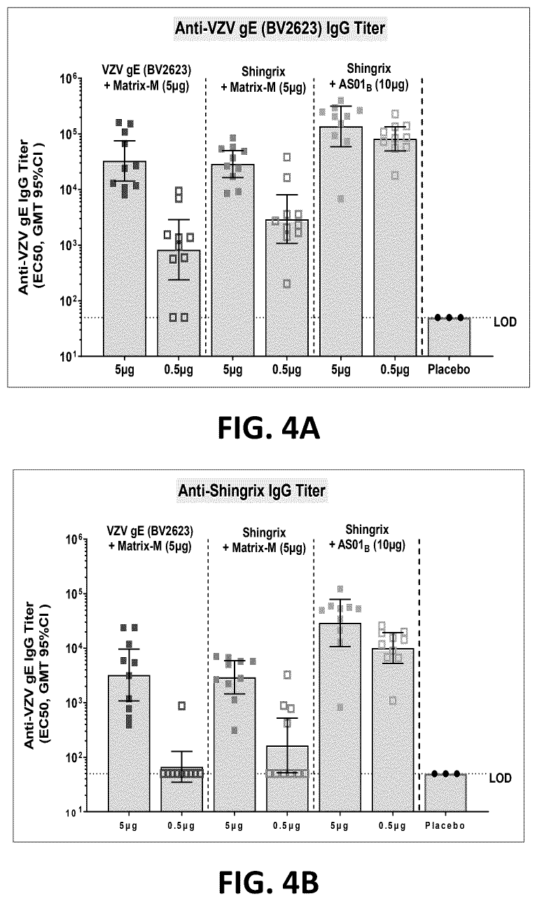

FIGS. 4A and 4B show the anti-VZV gE IgG responses from an ELISA assay of the mouse serum obtained at Day 35. FIG. 4A shows the results for Anti-VZV gE (BV2623) IgG titer, while FIG. 4B shows results for the Anti-Shingrix IgG titer. Each bar graph shows results obtained after administration of VZV gE (BV2623)+Matrix M at 5 μg and 0.5 μg, Shingrix+Matrix M at 5 μg and 0.5 μg, and Shingrix+AS01B.

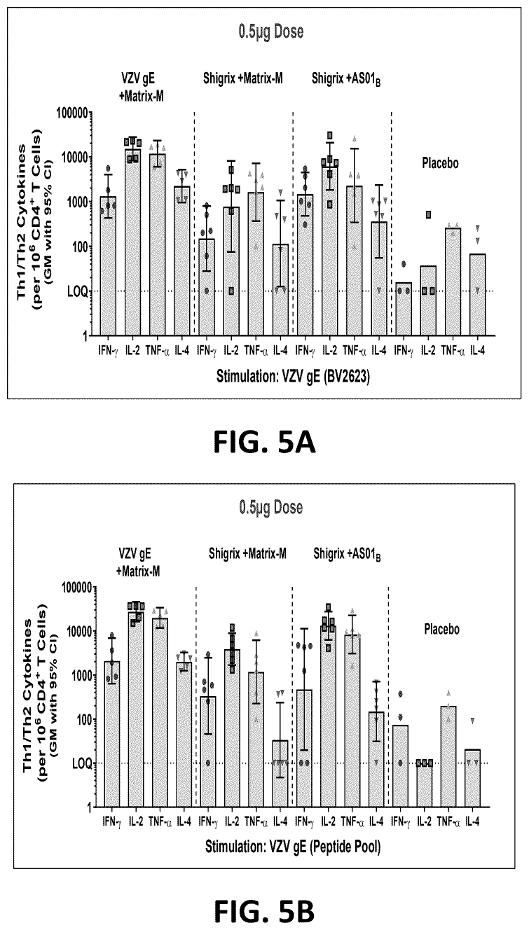

FIGS. 5A and 5B show effector memory CD4+ T cell Th1/Th2 cytokine responses in mouse spleen following stimulation with BV2623 (FIG. 5A) versus peptide pool gE (FIG. 5B). Each graph shows the cytokine response for each of the vaccine mixtures at a 0.5 μg dose and for placebo. The results from vaccination with VZV gE (BV2623)+Matrix-M are shown in the figures as “VZV gE+Matrix-M.” Cytokine responses shown for IFN-γ, IL-2, TNF-α, and IL-4. When stimulation was by either BV2623 or gE peptide pool, the BV262+Matrix-M combination induced similar or higher numbers of each of the cytokines than did Shingrix+AS01_{B}.

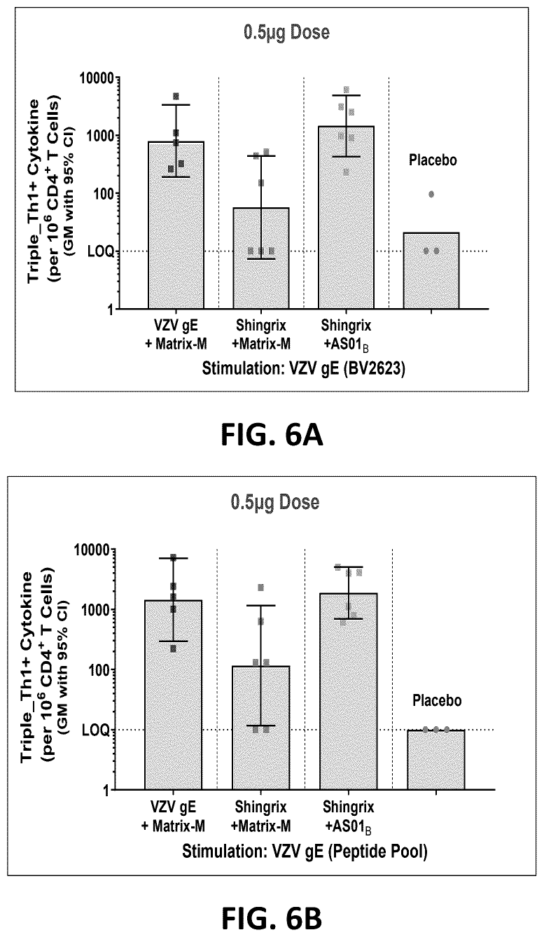

FIGS. 6A and 6B show triple Th1+ cytokine counts in mouse spleen following stimulation with BV2623 (FIG. 6A) versus peptide pool gE (FIG. 6B). Each graph shows the cytokine response for each of the vaccine mixtures at a 0.5 μg dose and for placebo. In each case, the Shingrix+Matrix-M vaccine resulted in lower counts than the Shingrix+AS01_{B} vaccine. However, the BV2623+Matrix-M vaccine resulted in counts having the same order of magnitude as the Shingrix+AS01_{B} vaccine.

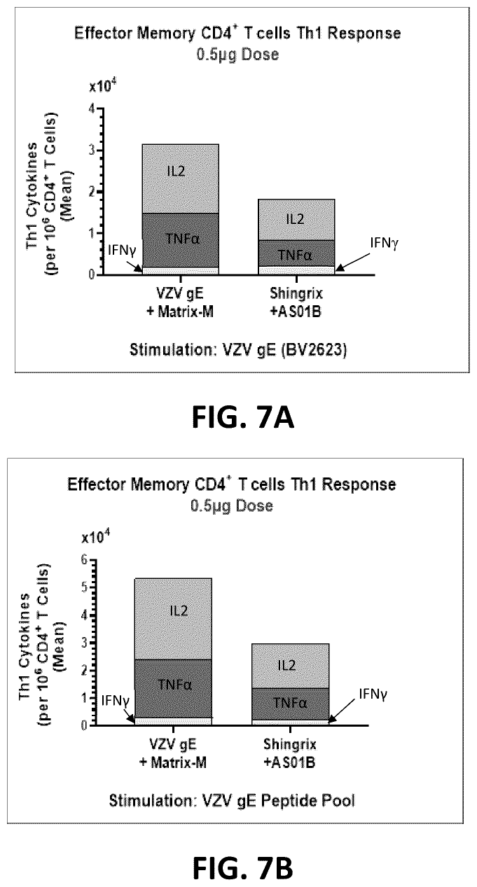

FIGS. 7A and 7B report effector memory CD4+ T cell Th1 response results, shown as cumulative bar graphs that include each of the cytokines IFNγ, TNFα and IL2, following stimulation with BV2623 (FIG. 7A) versus peptide pool gE (FIG. 7B). Results are shown only for spleens where the mice were administered VZV gE (BV2623)+Matrix M and Shingrix+AS01_{B}.

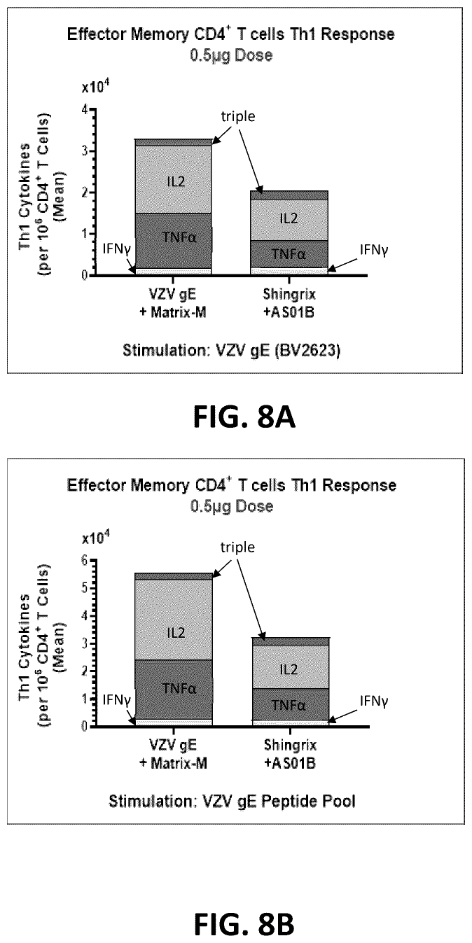

FIGS. 8A and 8B report effector memory CD4+ T cell Th1 response results, shown as cumulative bar graphs that include each of the cytokines IFNγ, TNFα, IL2, and triple following stimulation with BV2623 (FIG. 8A) versus peptide pool gE (FIG. 8B). Results are shown only for spleens where the mice were administered VZV gE (BV2623)+Matrix M and Shingrix+AS01_{B}.

These results confirm that the VZV gE (BV2623)+Matrix-M vaccine is producing an immunogenic effect in mice that is comparable to that produced by Shingrix+AS01_{B}.

### Example 3: Comparative Immunogenicity of New gE and gE/gI Constructions and GSK Shingrix Vaccines in Mice

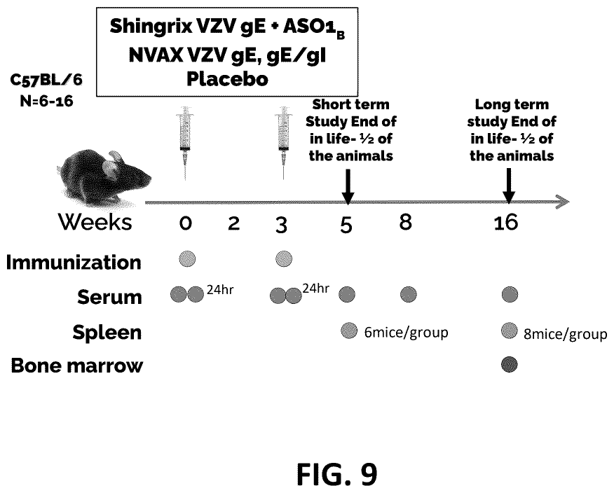

A longer-term study was made comparing the immunogenicity of the BV2623 and the BV2548 constructions to that of GSK's Shingrix vaccine, in mice. The timeline of the study is shown in FIG. 9. C57BL/6 mice were administered the vaccine on at weeks 0 and 3 of the study, e.g. on a Monday of week 0 and a Monday three weeks later. Serum was taken from the mice just before and 24 hours after administration of the vaccine, and at weeks 5, 8, and 16. The spleens of 6 mice per group were harvested at week 5 and of 8 mice per group at week 16. Bone marrow was harvested from 8 mice per group at week 16.

The overall health of the mice and particularly the injection sites were monitored to maintain good health. Blood serum was tested for pro-inflammatory cytokines 24 hours post injection using a LEGENDplex™ Mouse Anti-Virus Response Panel (13-plex) multiplex assay based on fluorescent-encoded beads that allows simultaneous quantification of 13 mouse proteins. Blood serum was used to determine anti-VZV gE, gE/gI and gI responses using ELISA. Spleens were used to measure CD4+ and CD8_ T cell responses using an ICCS assay. The large number of animals involved prevented the spleens from being obtained all on the same day, so they were obtained on days 112, 114, 119, and 121. An enzyme-linked immunosorbent spot (ELISpot) assay was used on bone marrow for measuring cytokine production in long-lived plasma cells.

The table below shows the number of groups of mice in each group and what they were administered.

TABLE 2
Combinations of vaccine (antigen) and adjuvant
used in the experiment of Example 3

| | | | Vaccine | | Adjuvant |
|---|---|---|---|---|---|
| Group | N | Vaccine | Dose (μg) | Adjuvant | Dose (μg) |
| 1 | 16 | Shingrix (gE) | 5 | AS01_{B} | 10 |
| 2 | 16 | Shingrix (gE) | 0.5 | AS01_{B} | 10 |
| 3 | 16 | gE (BV2623) | 5 | Matrix-M | 5 |
| 4 | 16 | gE (BV2623) | 0.5 | Matrix-M | 5 |
| 5 | 16 | gE/gI (BV2548) | 5 | Matrix-M | 5 |
| 6 | 16 | gE/gI (BV2548) | 0.5 | Matrix-M | 5 |
| 7 | 16 | gE/gI (BV2548) | 5 | AS01_{B} | 10 |
| 8 | 16 | gE/gI (BV2548) | 0.5 | AS01_{B} | 10 |
| 9 | 6 | Placebo | | | |

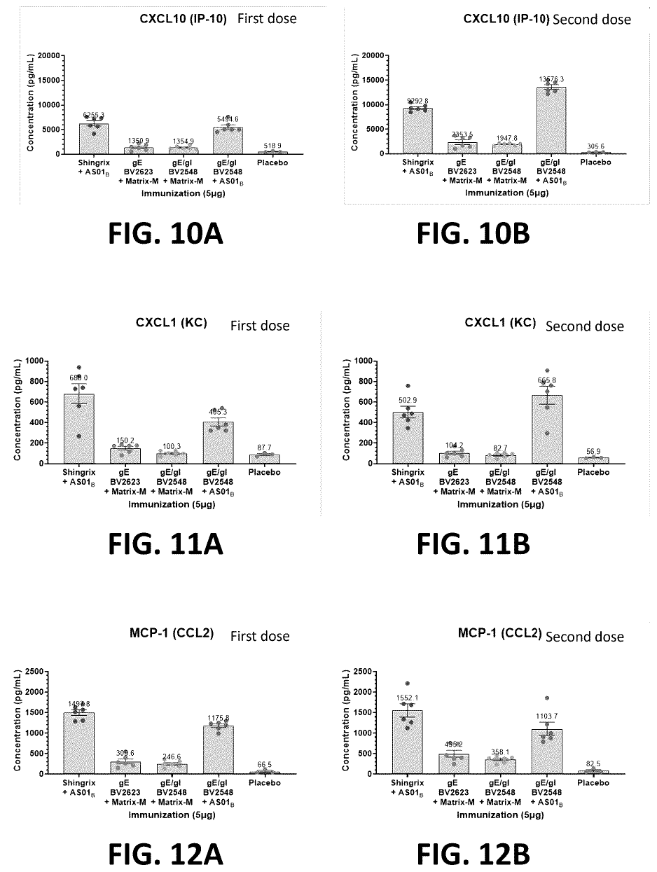

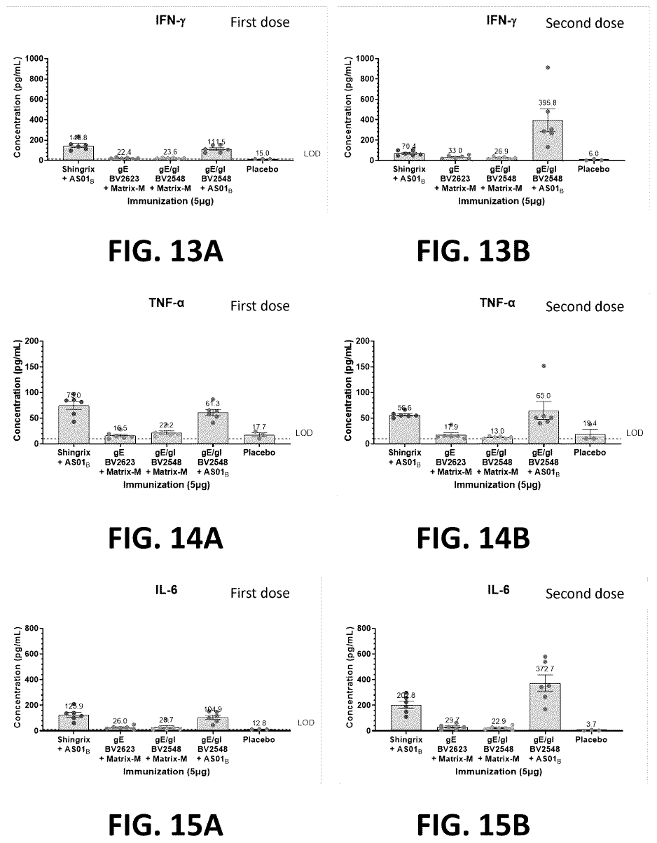

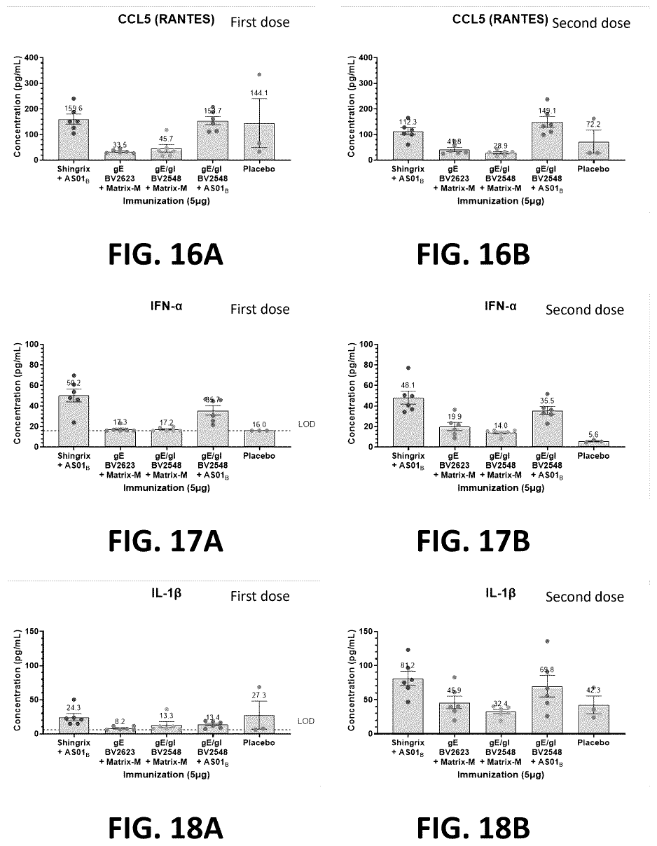

The LEGENDplex™ assay was used to measure the presence of proinflammatory cytokines resulting from administration of the vaccine. Nine out of the 13 cytokines measured in the assay showed measurable cytokine levels. FIGS. 10-18 present the results of nine representative cytokine measurements, respectively CXCL10 (IP-10), CXCL1 (KC), MCP-1 (CCL2), IFN-γ, TNF-α, IL-6, CCL5, IFN-α, IL-1β, for each of the four vaccine combinations and placebo. The assay was performed on blood taken 24 hours after administration of the first 5 μg dose and after the second 5 μg dose. For each cytokine, the first figure shows the results obtained following the first dose and the second figure shows the results obtained following the second dose. For example, FIG. 10A shows the results obtained for CXCL (IP-10) after the first dose and FIG. 10B shows the results obtained for CXCL (IP-10) after the second dose.

In nearly all cases, the cytokine concentration was relatively high for the two vaccines that used the AS01_{B} adjuvant (Shingrix and gE/gI (BV2548)), while it was relatively low for the vaccines using Matrix-M as the adjuvant (both the gE (BV2623) and gE/gI (BV2548) antigens), in some cases almost approaching the same level as the placebo.

Below are tables that summarize the first and second dose results shown in FIGS. 10-18, listing the average and the standard error of the mean (SEM).

TABLE 3
Average and SEM counts for various blood-borne cytokines found 24 hours after
administration of the first dose.

| | CXCL10 | CXCL1 | MCP-1 | | | | | | |
|---|---|---|---|---|---|---|---|---|---|
| Vaccine | (IP10) | (KC) | (CCL2) | IFN-γ | TNF-α | IL-6 | CCL-5 | INF-α | IL-1b |
| Shingrix + | 6,255.0 | 680.0 | 1,498.0 | 146.8 | 75.1 | 125.9 | 159.6 | 50.3 | 24.3 |
| _AS01_{B} | (541.9) | (97.9) | (70.2) | (19.9) | (8.3) | (20.4) | (19.8) | (6.4) | (5.4) |
| gE | 1,351.0 | 150.2 | 309.6 | 22.4 | 16.5 | 26.0 | 33.5 | 17.4 | 8.2 |
| (BV2623) + | (256.0) | (17.6) | (59.2) | (2.7) | (2.8) | (5.1) | (3.0) | (1.2) | (1.1) |
| MM | | | | | | | | | |
| gE/GI | 1,355.0 | 100.3 | 246.6 | 23.6 | 22.2 | 28.7 | 45.7 | 17.2 | 13.3 |
| (BV2548) + | (142.9) | (9.1) | (33.6) | (2.0) | (3.4) | (12.0) | (15.4) | (0.7) | (4.7) |
| MM | | | | | | | | | |
| gE/GI | 5,495.0 | 405.3 | 1,176.0 | 111.5 | 61.3 | 104.9 | 153.7 | 35.8 | 13.4 |
| (BV2548) + | (470.0) | (41.0) | (45.8) | (14.1) | (6.3) | (17.2) | (16.0) | (4.6) | (2.0) |
| MM | | | | | | | | | |
| Placebo | 518.9 | 87.7 | 66.5 | 15.0 | 17.7 | 12.8 | 48.9# | 16.0 | 27.3 |
| | (67.0) | (10.0) | (25.2) | (0.0) | (4.1) | (0.8) | (NA) | (—) | (20.6) |

TABLE 4
Average and SEM counts for various blood-borne cytokines found 24 hours after
administration of the second dose.

| | CXCL10 | CXCL1 | MCP-1 | | | | | | |
|---|---|---|---|---|---|---|---|---|---|
| Vaccine | (IP10) | (KC) | (CCL2) | IFN-γ | TNF-α | IL-6 | CCL-5 | INF-α | IL-1b |
| Shingrix + | 9,293 | 502.9 | 1,552 | 70.4 | 56.6 | 202.8 | 112.3 | 48.1 | 81.2 |
| _AS01_{B} | (294.7) | (57.5) | (163.1) | (9.95) | (2.3) | (28.4) | (14.2) | (6.4) | (10.7) |
| gE | 2,354 | 104.2 | 495.2 | 33.0 | 17.9 | 29.7 | 41.8 | 19.9 | 45.9 |
| (BV2623) + | (470) | (16.5) | (85.9) | (7.0) | (3.96) | (7.9) | (8.3) | (4.0) | (9.2) |
| MM | | | | | | | | | |
| gE/GI | 1,948 | 82.7 | 358.1 | 26.9 | 12.96 | 22.9 | 28.9 | 14 | 32.4 |
| (BV2548) + | (95.0) | (9.6) | (34.1) | (4.3) | (0.95) | (5.5) | (4.5) | (1.3) | (3.2) |
| MM | | | | | | | | | |
| gE/GI | 13,576 | 665.8 | 1,104 | 395.8 | 65.0 | 372.7 | 149.1 | 35.6 | 69.8 |
| (BV2548) + | (468.7) | (88.6) | (161.9) | (112.9) | (17.5) | (64.7) | (21.1) | (3.9) | (15.9) |
| MM | | | | | | | | | |
| Placebo | 305.6 | 56.9 | 82.5 | 5.97 | 19.4 | 3.7 | 72.2 | 5.6 | 42.3 |
| | (37.6) | (3.5) | (26.9) | (3.9) | (9.4) | (1.6) | (44.9) | (0.8) | (13.2) |

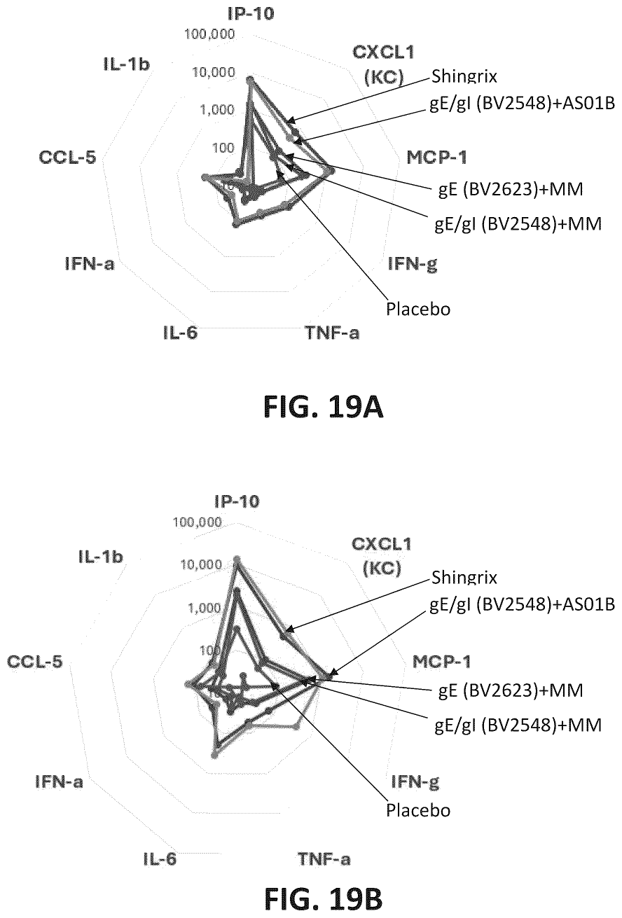

FIGS. 19A and 19B show cytokine contour plots depicting the counts for each of the vaccine compositions and placebo, as set forth in Tables 3 and 4 respectively.

FIG. 20 shows the CD4+ T cell response following stimulation using the VZV gE peptide pool for four different cytokines, IFNγ, TNFα, IL2 and IL4, across all mouse groups. The y-axis corresponds to Th1/Th2 cytokine counts (per 10⁶ CD4+ T cells). Under each cytokine are nine bars, which correspond to mouse groups 1-9 in order. For example, under IFNγ, the bar furthest to the left corresponds to mouse group 1 (5 μg Shingrix+AS01_{B}) and the bar furthest to the right corresponds to mouse group 9 (placebo). The immune response for mouse groups 7 and 8 (5 μg and 0.5 μg gE/gI+AS01_{B}) is increased relative to groups 1 and 2 (5 μg and 0.5 μg Shingrix+AS01_{B}).

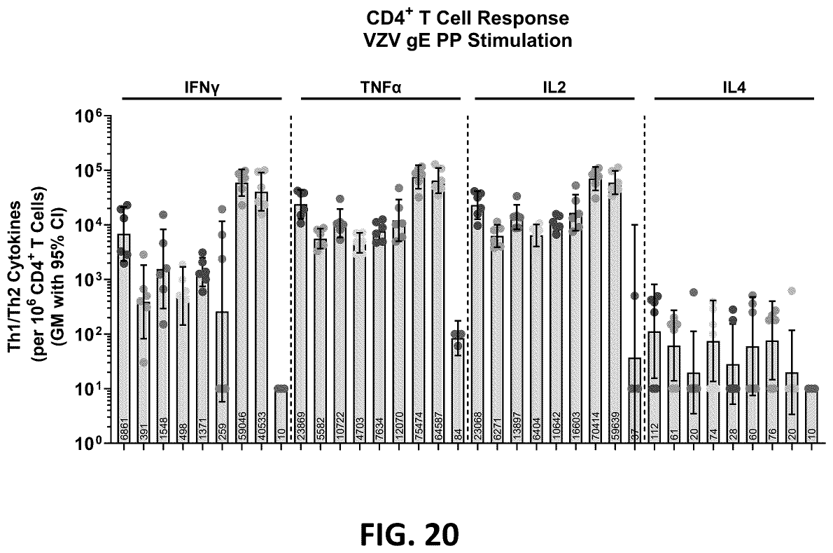

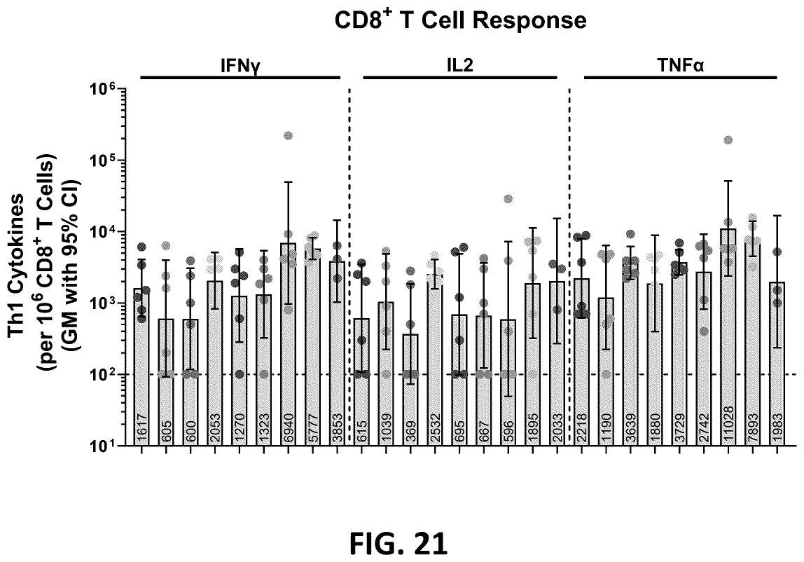

FIG. 21 shows the CD8⁺ T cell response following stimulation using the VZV gE peptide pool for three different cytokines, IFNγ, IL2, and TNFα, across all mouse groups. The y-axis corresponds to Th1/Th2 cytokine counts (per 10⁶ CD8+ T cells). Under each cytokine are nine bars, which correspond to mouse groups 1-9 in order. For example, under IFNγ, the bar furthest to the left corresponds to mouse group 1 (5 μg Shingrix+AS01_{B}) and the bar furthest to the right corresponds to mouse group 9 (placebo). The immune response for mouse groups 7 and 8 (5 μg and 0.5 μg gE/gI+AS01_{B}) tend to be higher compared to groups 1 and 2 (5 μg and 0.5 μg Shingrix+AS01_{B}).

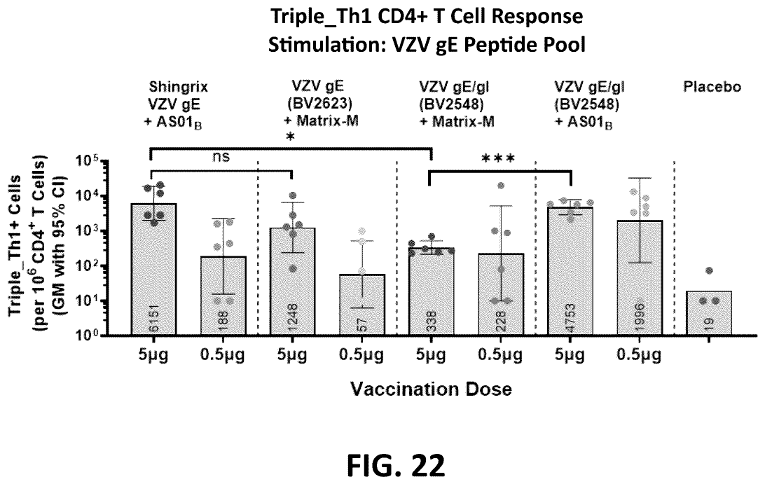

FIG. 22 shows Triple Th1 CD4⁺ Th1 responses following stimulation using the VZV gE peptide pool, measured 14 days after the second immunization, for each of the 9 mouse groups. The y-axis corresponds to the number of triple Th1+ T cells per 10⁶ CD4+ T cells. Mouse group 1 (Shingrix+AS01_{B}) shows the highest response (6151), with VZV gE/gI (BV2548)+AS01_{B} (4753) close behind in count.

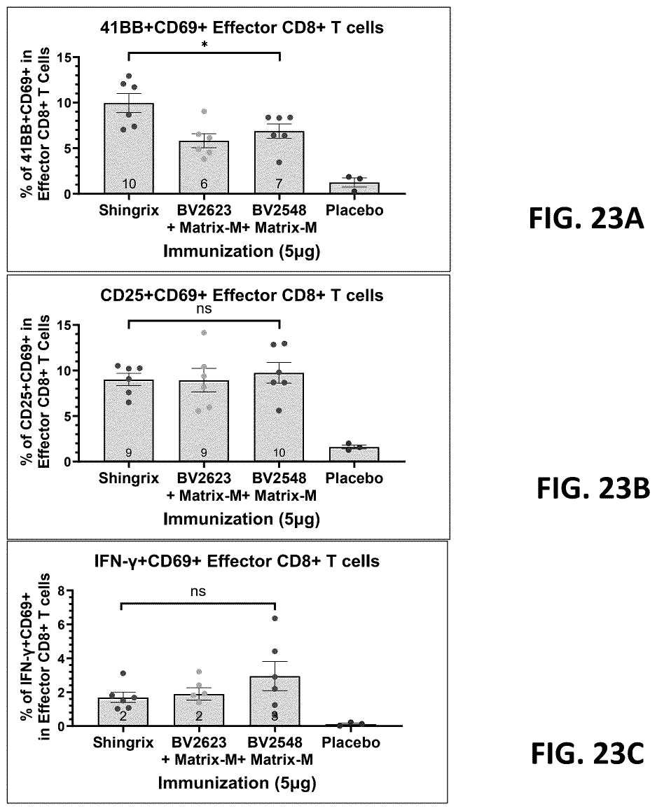

FIGS. 23A-23C show a summary of antigen-specific CD8+ T cell responses measured 14 days after the second immunization, for mouse groups 1, 3, 5, and 9 (Shingrix (gE)+AS01_{B}, BV2623 (gE)+Matrix-M, BV2548 (gE/gI)+Matrix-M, and placebo. FIG. 23A shows the percentage of 41BB+CD69+ in effector CD8+ T cells. FIG. 23B shows the percentage of CD25+CD69+ in effector CD8+ T cells. FIG. 23C shows the percentage of IFN-γ+CD69+ in effector CD8+ T cells.

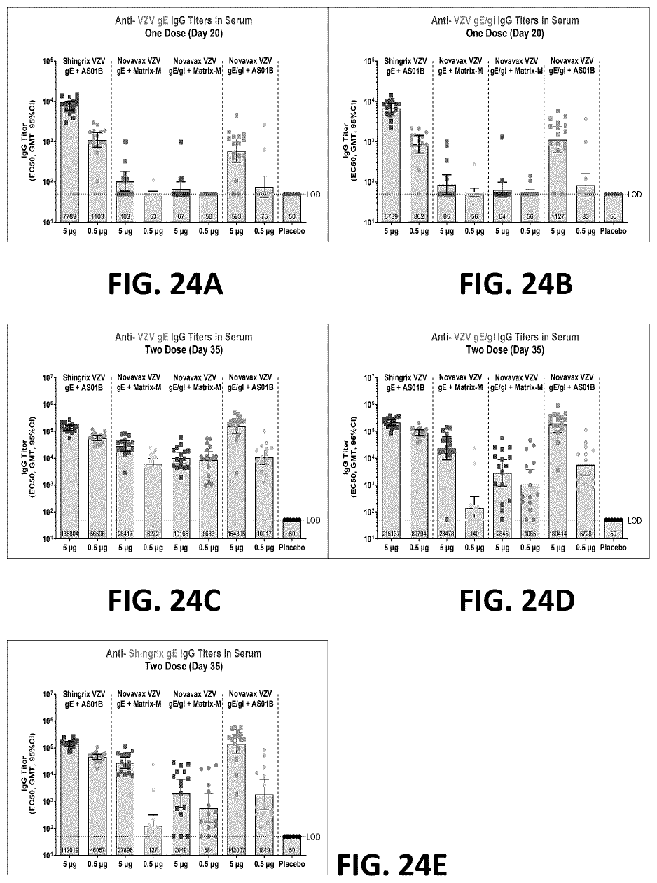

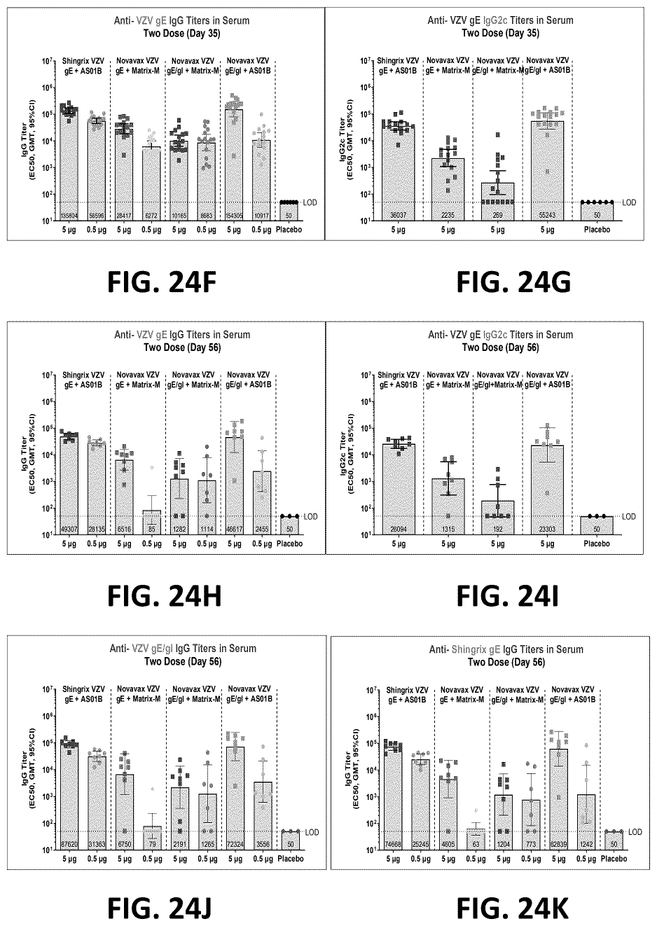

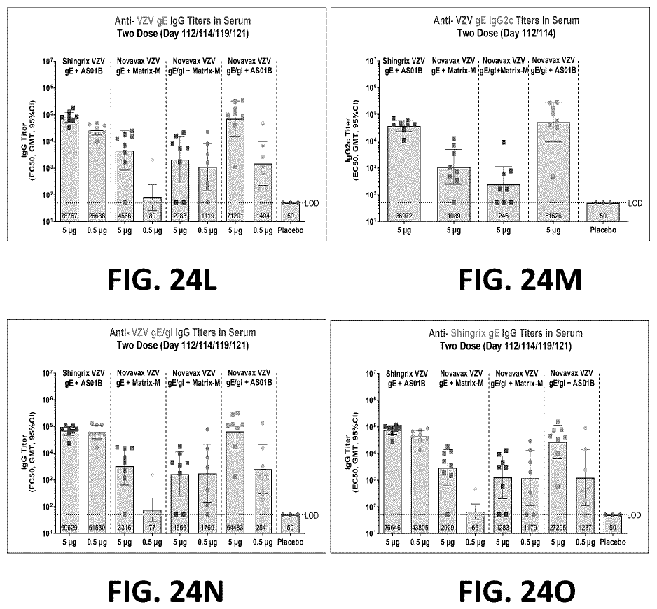

FIGS. 24A-24O show Anti-VZV gE or gE/gI IgG titers at different points throughout the experiment, for each of the mouse groups unless indicated. FIGS. 24A and 24B respectively show anti-VZV gE and anti-VZV gE/gI IgG titers in serum after one immunization dose (Day 20). FIGS. 24C and 24D respectively show anti-VZV gE and anti-VZV gE/gI IgG titers in serum after two immunization doses (Day 35). FIG. 24E shows anti-Shingrix gE IgG titers in serum after two immunization doses (Day 35). FIG. 24F shows anti-VZV gE IgG titers in serum after two immunization doses (Day 35), while FIG. 24G shows anti-VZV gE IgG2c titers in serum after two immunization doses (Day 35) for mouse groups 1, 3, 5, 7, and 9. FIG. 24H shows anti-VZV gE IgG titers in serum after two immunization doses (Day 56), while FIG. 24I shows anti-VZV gE IgG2c titers in serum after two immunization doses (Day 56) for mouse groups 1, 3, 5, 7, and 9. FIG. 24J shows anti-VZV gE/gI IgG titers in serum after two immunization doses (Day 56) across all mouse groups while FIG. 24K shows anti-Shingrix (gE) IgG titers in serum after two immunization doses (Day 56) across all mouse groups. FIG. 24L shows anti-VZV gE IgG titers across all mouse groups at Day 112/114/119/121, while FIG. 24M shows anti-VZV gE IgG2c titers for mouse groups 1, 3, 5, 7, and 9 at Day 112/114. FIG. 24N shows anti-VZV gE/gI IgG titers in serum after two immunization doses (Day 112/114/119/121 across all mouse groups while FIG. 24O shows anti-Shingrix (gE) IgG titers in serum after two immunization doses (Day 112/114/119/121 across all mouse groups.

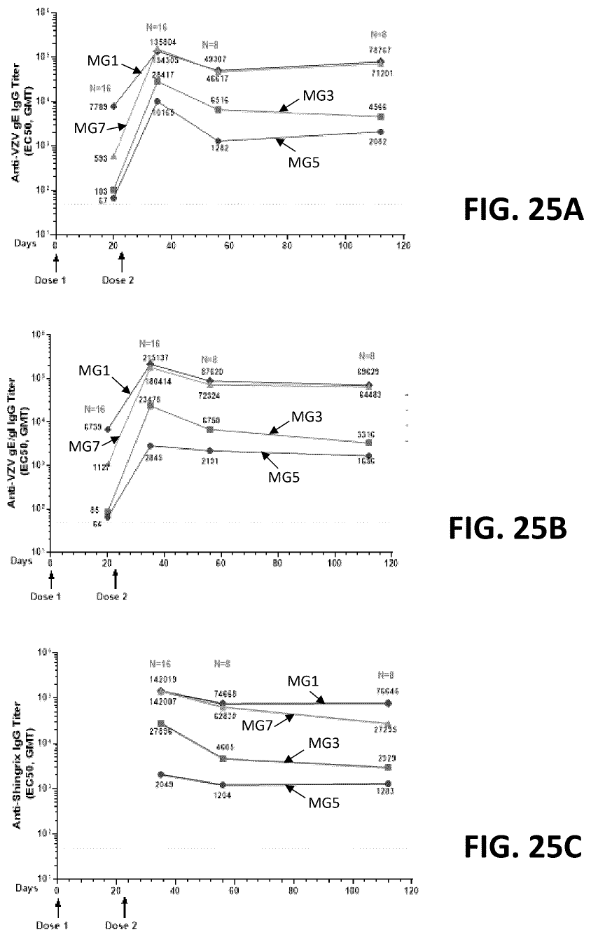

FIGS. 25A-25C show the time development and durability of IgG arising from various antigens as function of time over the experimental period, for mouse groups 1 (MG1), 3 (MG3), 5 (MG5), and 7 (MG7) (5 μg doses of antigen). FIG. 25A shows the anti-VZV gE IgG titer. FIG. 25B shows the anti-VZV gE/gI IgG titer. FIG. 25C shows the anti-Shingrix IgG titer.

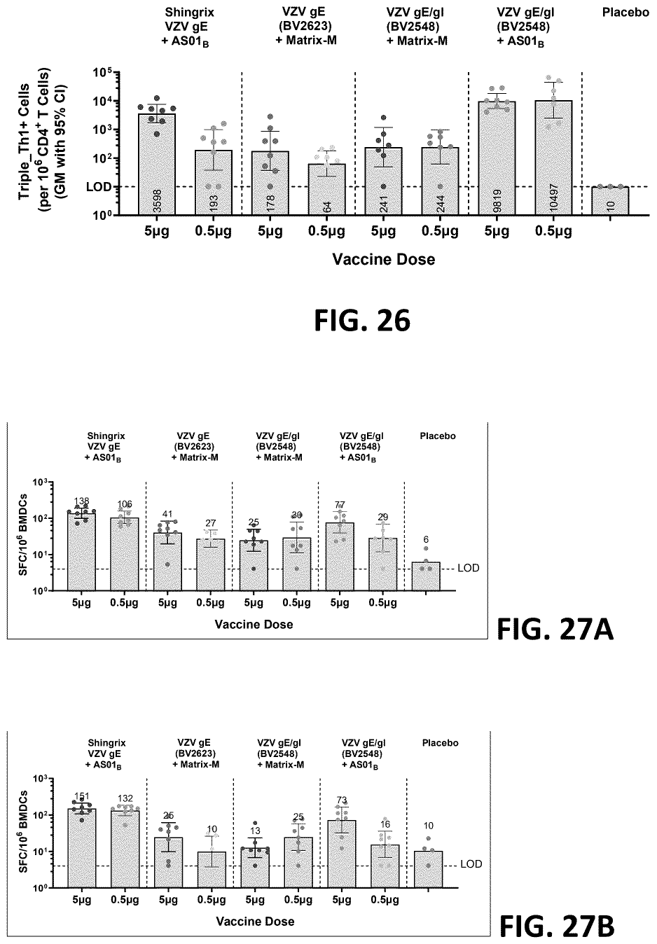

FIG. 26 shows the number of triple Th1+ cells per 10⁶ CD4+ T cells over all mouse groups when stimulated with the VZV gE peptide pool at 11 weeks after the second dose.

FIGS. 27A and 27 B show the numbers of antigen-specific bone marrow-derived memory B cells obtained using B cell ELISPOT at 11 weeks after the second immunization, over all mouse groups. The y-axes of the graphs show the numbers of spot-forming cells (SFC) per 10⁶ bone marrow-derived cells (BMDC). FIG. 27A shows the results coated with BC2623 (VZV gE) and FIG. 27B shows the results coated with Shingrix.

### Example 4: Dose Adjustment of gE/gI (BV2548) Constructions and GSK Shingrix Vaccines in Mice

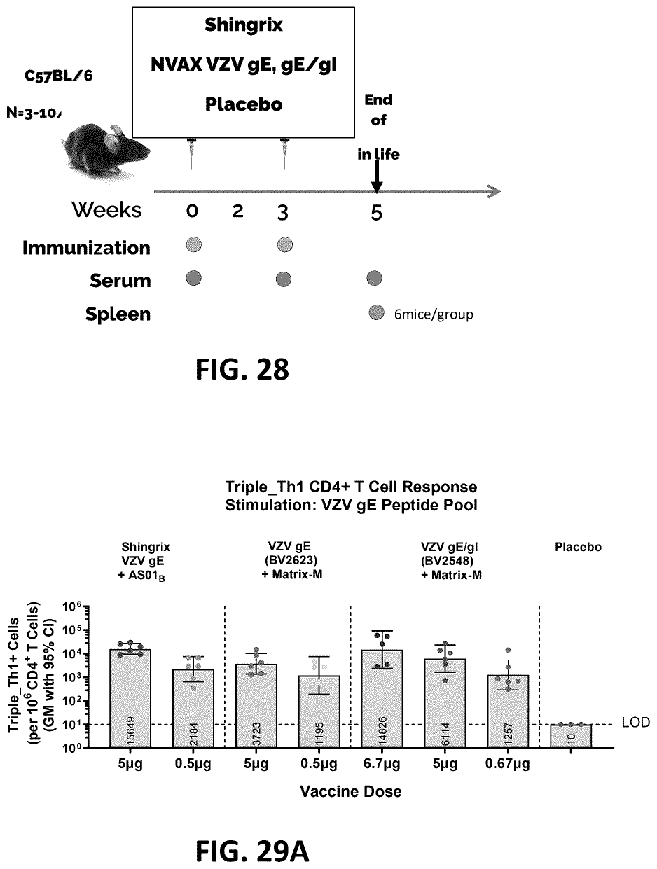

A study was made to confirm the immunogenicity of gE/gI with adjusted doses of antigen protein. The timeline of the study is shown in FIG. 28. C57BL/6 mice were administered the vaccine at weeks 0 and 3 of the study, e.g. on a Monday of week 0 and a Monday three weeks later. Serum was taken from the mice just before administration of the vaccine, at week 3, and at week 5. The spleens of 6 mice per group were harvested at week 5.

The overall health of the mice and particularly the injection sites were monitored to maintain good health. Blood serum was used to determine anti-VZV gE, gE/gI and gI responses using ELISA. Spleens were used to measure CD4+ and CD8_T cell responses using an ICCS assay.

The table below shows the number of mice in each group and what they were administered. It should be noted that the data from one mouse in group 5 was excluded from analysis due to abnormally low Th1 cytokine and IgG responses.

TABLE 5
Combinations of vaccine (antigen) and adjuvant
used in the experiment of Example 4

| | | | Vaccine | | Adjuvant |
|---|---|---|---|---|---|
| Group | N | Vaccine | Dose (μg) | Adjuvant | Dose (μg) |
| 1 | 10 | Shingrix (gE) | 5 | AS01_{B} | 10 |
| 2 | 10 | Shingrix (gE) | 0.5 | AS01_{B} | 10 |
| 3 | 10 | gE (BV2623) | 5 | Matrix-M | 5 |
| 4 | 10 | gE (BV2623) | 0.5 | Matrix-M | 5 |
| 5 | 10 | gE/gI (BV2548) | 6.7 | Matrix-M | 5 |
| 6 | 10 | gE/gI (BV2548) | 5 | Matrix-M | 5 |
| 7 | 10 | gE/gI (BV2548) | 0.67 | Matrix-M | 5 |
| 8 | 3 | Placebo | | | |

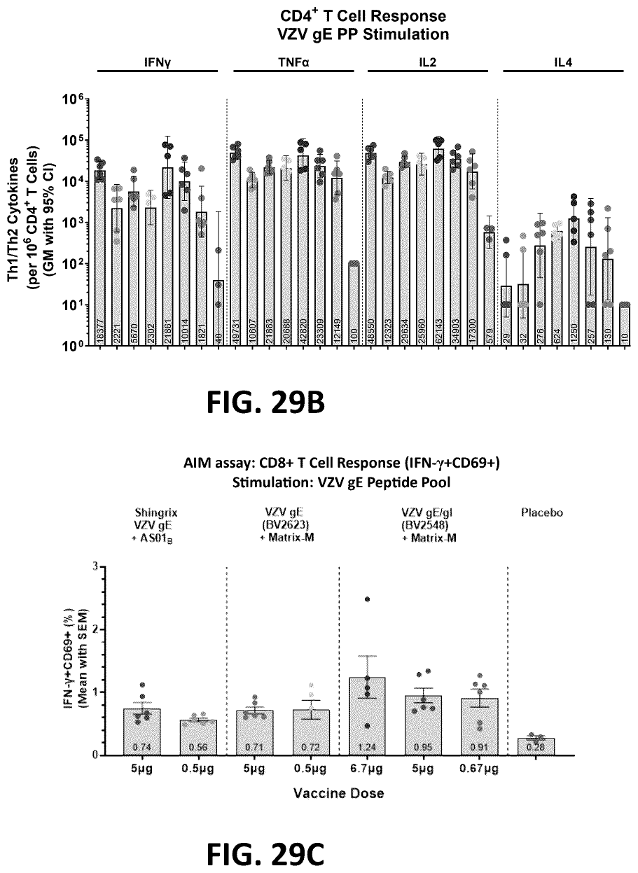

FIGS. 29A-29C show results of the immune response in the spleen. FIG. 29A shows the number of triple Th1 CD4+ T cells following stimulation with the VZV gE peptide pool, across all mouse groups. FIG. 29B shows the number of Th1/Th2 cytokines per 10⁶ CD4+ T cells for IFNγ, TNFα, IL2, and IL4, following stimulation with the VZV gE peptide pool. FIG. 29C illustrates the CD8+ T cell response by showing the percentage of IFNγ+CD69+ following stimulation with the VZV gE peptide pool.

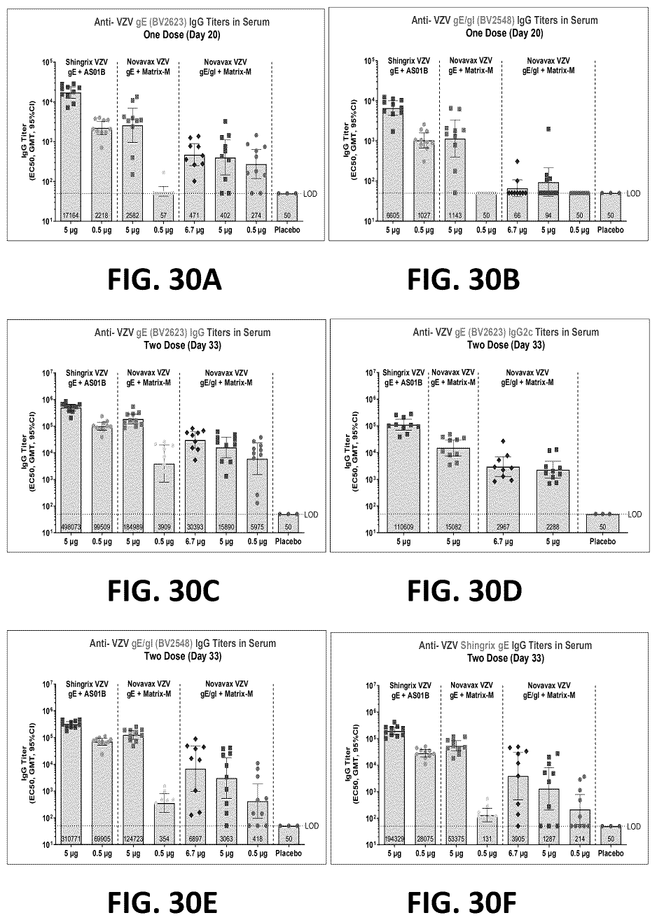

FIGS. 30A-30F show IgG titers in serum. FIG. 30A shows anti-VZV gE (BV2623) IgG titers after one dose (day 20) and FIG. 30B shows anti-VZV gE/gI (BV2548) IgG titers after one dose (day 20). FIG. 30C shows anti-VZV gE (BV2623) IgG titers after two doses (day 33) and FIG. 30D shows anti-VZV (BV2623) IgG2c titers after two doses (day 33). FIG. 30E shows anti-VZV gE/gI (BV2548) IgG titers after two doses (day 33) and FIG. 30F shows anti-Shingrix IgG titers after two doses (day 33).

Various modifications, equivalent processes, as well as numerous structures to which the present invention may be applicable will be readily apparent to those of skill in the art to which the present invention is directed upon review of the present specification. The claims are intended to cover such modifications and devices.

As noted above, the present invention is applicable to immunological compositions for VZV vaccines. Accordingly, the present invention should not be considered limited to the particular examples described above, but rather should be understood to cover all aspects of the invention as fairly set out in the attached claims.

1. An immunogenic composition for stimulating an immune response against varicella zoster, comprising an effective amount of recombinant varicella zoster virus (VZV) glycoprotein, wherein the VZV glycoprotein comprises VZV glycoprotein E (VZV gE) complexed with VZV glycoprotein I (VZV gI).

2. The immunogenic composition of claim 1, wherein the VZV gE has at least 90% homology to the amino acid sequence as set forth in SEQ ID No: 2.

3. The immunogenic composition of claim 1, wherein the VZV gE has at least 90% homology to the amino acid sequence as set forth in SEQ ID No: 9.

4. The immunogenic composition of claim 1, further comprising a saponin-based adjuvant.

5. The immunogenic composition of claim 4, wherein the saponin-based adjuvant comprises AS01_{B}.

6. The immunogenic composition of claim 4, wherein the saponin-based adjuvant comprises Matrix-A and Matrix-C present in a weight ratio of between 80:20 and 90:10.

7. The immunogenic composition of claim 6, wherein the saponin-based adjuvant comprises Matrix-A and Matrix-C present in a weight ratio of 85:15.

8. The immunogenic composition of claim 4, wherein the saponin-based adjuvant is present in a dose of about 5 μg to about 200 μg.

9. The immunogenic composition of claim 1, wherein the recombinant varicella zoster virus glycoprotein is present at about 0.1 μg to 200 μg.

10. The immunogenic composition of claim 1, further comprising a pharmaceutically acceptable buffer.

11. A method of stimulating an immune response against varicella zoster virus, comprising administering the immunogenic composition of claim 1 to a subject.

12. The method of claim 11, wherein the composition is administered to the subject through at least one of subcutaneous, intraperitoneal, intravenous, intraarterial, intramuscular, or intrasternal injection, intravenous, or kidney dialytic infusion techniques.

13. A prefilled syringe comprising the immunogenic composition of claim 1.

14. A kit comprising a first vial containing the immunogenic composition of claim 1 and a second vial containing a saponin-based adjuvant.

15. The kit of claim 14, wherein the first vial includes space to receive contents of the second vial or the second vial includes space to receive contents of the first vial.

16. An immunogenic composition for stimulating an immune response against varicella zoster, comprising an effective amount of at least one recombinant varicella zoster virus glycoprotein, and a saponin-based adjuvant comprising Matrix-A and Matrix-C.

17. The immunogenic composition of claim 16, wherein the varicella zoster virus glycoprotein comprises recombinant varicella zoster virus glycoprotein E (VZV gE).

18. The immunogenic composition of claim 17, wherein the recombinant VZV gE has at least 90% homology to the amino acid sequence as set forth in SEQ ID No: 4.

19. The immunogenic composition of claim 16, wherein the varicella zoster virus glycoprotein comprises varicella zoster virus glycoprotein E (VZV gE) complexed with varicella zoster virus glycoprotein I (VZV gI).

20. The immunogenic composition of claim 19, wherein the recombinant VZV gE comprises an extracellular region having at least 90% homology to the amino acid sequence as set forth in SEQ ID No: 2.

21. The immunogenic composition of claim 19, wherein the recombinant VZV gE comprises an extracellular region having at least 90% homology to the amino acid sequence as set forth in SEQ ID No: 9.

22. The immunogenic composition of claim 16, wherein the saponin-based adjuvant comprises Matrix-A and Matrix-C present in a weight ratio of 85:15.

23. The immunogenic composition of claim 22, wherein the saponin-based adjuvant is a dose of about 5 μg to about 200 μg.

24. The immunogenic composition of claim 16, wherein the recombinant varicella zoster virus glycoprotein is present at about 0.1 μg to 200 μg.

25. The immunogenic composition of claim 16, further comprising a pharmaceutically acceptable buffer.

26. A method of stimulating an immune response against varicella zoster virus comprising administering the immunogenic composition of claim 16 to a subject.

27. The method of claim 26, wherein the composition is administered to the subject through at least one of subcutaneous, intraperitoneal, intravenous, intraarterial, intramuscular, or intrasternal injection, intravenous, or kidney dialytic infusion techniques.

28. A prefilled syringe comprising the immunogenic composition of claim 16.

29. A kit comprising a first vial containing an effective amount of at least one recombinant varicella zoster virus glycoprotein and a second vial containing a saponin-based adjuvant comprising Matrix-A and Matrix-C.

30. The kit of claim 29, wherein the first vial includes space to receive contents of the second vial or the second vial includes space to receive contents of the first vial.
